(19) United States
(12) Patent Application Publication
(10) Pub. No.: US 2020/0155608 A1
(43) Pub. Date: May 21, 2020

(54) Therapeutic Treatments Via Intravenous Infusion Of Mesenchymal Stem Cells

(71) Applicants: E. Jeffrey Donner, Johnstown, CO (US); Kenneth A. Pettine, Fort Collins, CO (US)

(72) Inventors: E. Jeffrey Donner, Johnstown, CO (US); Kenneth A. Pettine, Fort Collins, CO (US)

(21) Appl. No.: 16/197,105

(22) Filed: Nov. 20, 2018

(51) Int. Cl. A61K 35/28; A61K 9/00; A61P 37/00; A61P 19/00; A61P 1/16; C12N 5/0775

(52) U.S. Cl. CPC A61K 35/28; A61K 9/0019; C12N 5/0663; A61P 19/00; A61P 1/16; A61P 37/00

(57) ABSTRACT

The present novel technology relates to an autologous or allogeneic mesenchymal stem cell (MSC) therapy that comprises a pre-determined amount of a processed bone marrow cellular matrix with a pre-determined amount of pre-mixture, where the pre-mixture includes quantities of anticoagulant solution, dextrose and phosphate buffered saline, and methods for production. The present novel technology further relates to methods of use of the therapy to treat various autoimmune diseases, alcohol related liver pathology, and acute soft tissue injuries, and the like, via intravenous infusion of the MSCs.

### FIELD OF THE INVENTION

The present disclosure relates to therapeutic products that contain mesenchymal stem cells (MSCs), said therapeutic products being useful for treating autoimmune conditions and for promoting rapid healing from acute traumatic soft tissue orthopedic injuries, and the like. The disclosure also relates to methods of use of said MSC-containing therapeutic products for treatment of autoimmune conditions and for promoting rapid healing from acute traumatic soft tissue orthopedic injuries.

### BACKGROUND AND SUMMARY OF THE INVENTION

MSCs, after their initial discovery in bone marrow, have been isolated and characterized from several adult and fetal tissues, including adipose (fat), dermis (skin), synovial fluid, periosteum, umbilical cord blood, placenta, and amniotic fluid. MSCs are partially defined by their ability to differentiate into tissues including osteoblasts (bone cells), chondrocytes (cartilage cells), myocytes (muscle cells), and adipocytes (fat cells). But it is believed that it is their trophic, paracrine, and immunomodulatory functions that may have the greatest therapeutic impact in vivo. Unlike pharmaceutical treatments that deliver a single agent at a specific dose, MSCs are site regulated and secrete bioactive factors and signals at variable concentrations in response to local microenvironmental cues. Significant progress has been made in understanding the biochemical and metabolic mechanisms and feedback associated with MSC response. The anti-inflammatory and immunomodulatory capacity of MSC may be paramount in the restoration of localized or systemic conditions for normal healing and tissue regeneration. Allogeneic MSC treatments, categorized as a drug by regulatory agencies, have been widely pursued, but new studies demonstrate the efficacy of autologous MSC therapies, even for individuals affected by a disease state. Safety and regulatory concerns surrounding allogeneic cell preparations make autologous and minimally manipulated cell therapies an attractive option for many regenerative, anti-inflammatory, and autoimmune applications. (See, Murphy, M. B., et al., “Mesenchymal stem cells: environmentally responsive therapeutics for regenerative medicine,” Experimental & Molecular Medicine, 45: e54 (2013)). Nonetheless, allogeneic MSC treatments may prove valuable in certain situations, such as, illustratively, under circumstances where it is risky or not possible to harvest autologous MSCs.

The primary trophic property of MSCs is the secretion of growth factors and other chemokines to induce cell proliferation and angiogenesis. MSCs express mitogenic proteins such as transforming growth factor-alpha (TGF-α), TGF-ß, hepatocyte growth factor (HGF), epithelial growth factor (EGF), basic fibroblast growth factor (FGF-2) and insulin-like growth factor-1 (IGF-1) to increase fibroblast, epithelial and endothelial cell division. Vascular endothelial growth factor (VEGF), IGF-1, EGF and angiopoietin-1 are released to recruit endothelial lineage cells and initiate vascularization. It has been hypothesized that an individual's genotype has a role in the expression of and reaction to these cytokines, providing credence to the philosophy of personalized medicine utilizing responsive agents (that is, MSCs) rather than a dose of recombinant proteins or autologous growth factors (for example, platelet-rich plasma). The trophic effects extend beyond cell proliferation to the reduction of scar tissue formation presumably by local cells secreting paracrine factors keratinocyte growth factor, stromal cell-derived factor-1 (SDF-1), and macrophage inflammatory protein-1 alpha and beta.

Regarding the anti-inflammatory and immunomodulatory properties of MSCs, it is believed that, in many types of musculoskeletal trauma, inflammatory conditions at the site of injury impede the natural repair processes by local progenitor and mature cells. Without being bound by theory, it is believed that MSCs assist via paracrine mechanisms and modulate the regenerative environment via anti-inflammatory and immunomodulatory mechanisms. In response to inflammatory molecules such as interleukin-1 (IL-1), IL-2, IL-12, tumor necrosis factor-α (TNF-α) and interferon-gamma (INF-γ), MSCs secrete an array of growth factors and anti-inflammatory proteins with complex feedback mechanisms among the many types of immune cells. The key immunomodulatory cytokines include prostaglandin 2, TGF-ß1, HGF, SDF-1, nitrous oxide, indoleamine 2.3-dioxygenase, IL-4, IL-6, IL-10, IL-1 receptor antagonist and soluble tumor necrosis factor-α receptor. MSCs prevent proliferation and function of many inflammatory immune cells, including T-cells, natural killer cells, B-cells, monocytes, macrophages, and dendritic cells. Although MSCs across species are able to regulate T-cell activity, the mechanisms are not identical across mammalian species.

A characteristic of chronically inflamed environments is a persistent imbalance in the types of helper T-cells and macrophages. MSCs indirectly promote the transition of TH1 to TH2 cells by reducing INF-g and increasing IL-4 and IL-10. The restored TH1/TH2 balance has been shown to improve tissue regeneration in cartilage, muscle, and other soft tissue injuries, alleviate symptoms of autoimmune diseases, and have an anti-diabetic effect. Similarly, reduction in INF-γ and secretion of IL-4 promotes a shift in macrophages from M1 (pro-inflammatory, anti-angiogenic and tissue growth inhibition) to M2 (anti-inflammatory, pro-remodeling and tissue healing) type, an effect required for skeletal, muscular, and neural healing and regeneration. (See, Murphy, M. B., et al., “Mesenchymal stem cells: environmentally responsive therapeutics for regenerative medicine,” Experimental & Molecular Medicine, 45: e54 (2013)).

An embodiment of the invention provides therapeutic products that contain MSCs, and methods for intravenous (IV) use of said products for treating autoimmune conditions, for promoting rapid healing from acute traumatic soft tissue orthopedic injuries, for treating alcohol related liver pathology, and the like. In a primary aspect, the MSCs are contained in bone marrow concentrate (BMC) or adipose-derived stromal vascular fraction (SVF). The MSC is an adult stem cell found in the mammalian body, e.g., human body, throughout the vascular system (known as a pericyte) in adipose tissue and also in bone marrow. Currently, there is FDA regulatory risk associated with extracting MSCs found in adipose tissue. Additionally, it has not been demonstrated yet that pericytes located in one's blood vessels are capable of being harvested. Thus, it is currently believed that the most optimal area to source a large population of MSCs is from bone marrow or adipose tissue. The posterior iliac wing has been a primary location from which to extract bone marrow, since it provides the highest MSC concentration and is a standard technique for most physicians. The MSC is believed to be the most important adult stem cell in orthopedics and spine because it differentiates into chondroblasts, which in turn form cartilage; fibroblasts, which form ligaments, tendons, and muscles; or osteoblasts, which form bone. Orthopedic and spine surgeons have utilized the MSC for years in the form of bone grafts to promote healing of fractures and promote spinal fusion. However, it is believed that the most important characteristics of the MSC are what it promotes as itself, an adult stem cell.

The MSC has many functions, including powerful anti-inflammatory capabilities, ability to modulate the immune system, promote formation of new blood vessels (angiogenesis), and produce a wide range of growth factors, just to name a few of its attributes, as has been described by Murphy, M. B., et al. (“Mesenchymal Stem Cells: Environmentally Responsive Therapeutics for Regenerative Medicine,” Experimental & Molecular Medicine (2013) 45, e54; doi:10.1038/emm.2013.94 & 2013 KSBMB).

In another embodiment of the invention, disclosed herein are novel methods for utilizing BMC to treat chronic low back and neck pain related to damaged discs. It has been reported that utilizing BMC is standard of care in veterinary medicine, primarily in dogs and horses that have injured limbs. In another embodiment of the invention, disclosed herein are novel methods to systemically infuse MSCs intravenously (IV) to utilize the amazing functions of the MSC throughout the body. The following is a quote from an NIH manuscript, “Imagine a simple intravenous cell therapy that can restore function to damaged or diseased tissue, avoid host rejection and reduce inflammation throughout the body without the use of immunosuppressive drugs. Such a breakthrough would revolutionize medicine. Fortunately, pending regulatory approval, this approach might not be far off. Specifically, cell therapy utilizing adult MSCs, multipotent cells with the capacity to promote angiogenesis, differentiate to produce multiple types of connective tissue, and down-regulate an inflammatory process, is the focus of a multitude of clinical studies currently underway. MSCs are being explored to regenerate damaged tissue and treat inflammation resulting from: cardiovascular disease and myocardial infarction (MI), brain and spinal cord injuries, stroke, diabetes, cartilage and bone injury, Crohn's disease, and graft versus host disease. In this article, we highlight the recent paradigm shift which has occurred in therapeutic use of MSCs based on their immunomodulatory properties as opposed to their ability to differentiate into osteoblasts, chondroblasts, or fibroblasts.” (Ankrum, J., et al., “Mesenchymal Stem Cell Therapy: Two Steps Forward, One Step Back,” Trends Mol. Med., 2010, May 16(5):203-209. doi: 10.1016/j.molmed.2010.02.005; the disclosure of this Ankrum publication, and all publications cited therein, are hereby incorporated by reference in their entirety).

In one embodiment of the invention, disclosed herein are novel products and methods for utilizing MSC-containing BMC to accomplish treatment of the various conditions stated herein, wherein said treatment comprises IV administration. The embodiments disclosed herein are novel, even though there have appeared over 2,000 peer reviewed published papers concerning MSCs in just the last three years. The MSC is by far the most studied adult stem cell in the body. The main focus of all this research has been on the MSC's ability to influence biological function through its trophic mechanisms, including the secretion of cytokines which might serve both paracrine (cells communicating with adjacent cells) and endocrine (cells communicating long distances through the body) functions. (Iso Y, et al., “Multipotent Human Stromal Cells Improve Cardiac Function After Myocardial Infarction in Mice Without Long Term Engraftment,” Biochem Biophys Res Commun 2007; 354:700-706 [Pubmed: 17257581]). This shift away from just using MSCs in orthopedics and spine is believed to be the result of scientific observations that MSC therapy results in reduction of inflammation and apoptosis (cell death) in numerous disease models. It is believed that these positive systemic effects from the MSCs occur before they differentiate into osteoblasts, chondroblasts, or fibroblasts. Thus, it has been discovered that the MSC exerts these properties as a stem cell. (See Murphy publication above). This realization also resulted in a paradigm shift away from local injection of the MSCs into damaged tissue to systemic IV administration, which is less invasive and more convenient to the patient.

It has been reported (see Murphy publication above, and publications cited therein, all of which are hereby incorporated by reference in their entirety) that intravenous administration of MSCs would be expected to decrease the risk with cell therapy intervention for Multiple Sclerosis (MS) and/or Amyotrophic Lateral Sclerosis (ALS). Reportedly, ten MS patients received IV injection of autologous expanded bone marrow MSCs, resulting 10 months later in demonstrated improvement in visual acuity, evoked response latency and increased optic nerve area. Also, in a compassionate-use study of ex vivo cultured adult human MSCs (prochymal) for treatment of grade III and intravenous Graft-versus-host disease (GVHD), which is an autoimmune disease, 5/12 patients survived through follow-up at 611 days; whereas survival expectations are 5-10% if left to conventional treatment.

In another embodiment of the invention, disclosed herein are novel MSC-containing BMC or SVF products and IV methods for utilizing them in the treatments disclosed herein, wherein volumes of BMC or IVF injected via IV can range from about a few milliliters up to about 30 milliliters. In one aspect, the BMC product is put through a filter (Hemo-nate® Syringe Infusion Set) which removes the cell aggregates and particulates (18 micrometers or larger) prior to the slow infusion of the BMC. The infusion rate is about one c.c. of BMC or SVF per minute through a peripheral vein. The MSCs travel through the venous system and the first filter they encounter is the pulmonary capillary system. Without being bound by theory, it is believed herein that the MSCs trapped in pulmonary tissue eventually travel through capillaries into the arterial system and then travel throughout the body. Despite the MSCs temporary location in the pulmonary capillaries, numerous animal studies and some clinical trials have reported favorable outcomes following IV infusion of MSCs.

One embodiment of the invention is to provide MSC products and methods of their use intravenously in the treatment of a number of conditions. These conditions include, but are not limited to, the following: Parkinson's Disease, Multiple Sclerosis, Fibromyalgia, Rheumatoid Arthritis, Crohn's Disease, and the like, and even elevated liver enzymes from alcohol.

An embodiment of the disclosed invention provides formulations and methods for treating autoimmune conditions and promoting rapid healing from acute traumatic soft tissue orthopedic injuries. In one aspect, the MSCs can be utilized from BMC or from adipose SVF. In another aspect, the MSCs can also be utilized after cell expansion.

However, it is to be understood that, as contemplated herein, any suitable biologic MSC product may be utilized in the various embodiments of the invention disclosed herein. In a preferred embodiment, the biologic MSC product is BMC, because BMC is an excellent source of MSCs, which are stored in bone marrow in high concentrations. Illustrative examples of other sources of MSCs are peripheral blood, synovium, periosteum, skeletal muscle, and adipose tissue, and any other source of MSCs known to those skilled in the art.

MSCs obtained from BMC have been reported to possess many positive attributes. As stated earlier, MSCs are believed to be anti-inflammatory, secrete numerous growth factors, stimulate blood vessel formation, modulate the immune system to enhance healing, fight bacteria, turn into localized cells, and potentially heal inflammation. Accordingly, in one broad embodiment, disclosed herein is a method of treating autoimmune disorders and acute traumatic soft tissue orthopedic injuries.

Another embodiment of the disclosed invention provides formulations and methods for treating autoimmune conditions. It is known that MSCs are attracted by cytokines to areas of inflammation. When an MSC arrives in areas of bodily damage, they have been shown to actively participate in tissue repair. MSCs have the ability to suppress immune responses and have repeatedly shown efficacy in treating various autoimmune diseases. The proven ability of MSCs to regenerate damaged tissue combined with their capacity to regulate immune and inflammatory responses, gives a strong rationale for using MSCs as a new treatment option in autoimmune diseases. The treatment disclosed herein involves the intravenous infusion in the body of a patient of MSC-containing BMC.

MSCs are the preferred cells for the purpose of this invention. MSCs have been shown to have the potential to differentiate into several lineages including bone muscle, and stroma. Nonetheless, as contemplated herein, it should be understood that a variety of stem cells other than MSCs may be used.

All publications cited throughout this application are incorporated herein by reference in their entirety. Indeed, throughout this description, including the foregoing description of related art and cited publications, as well as any and all publications cited in what follows below, it is to be understood that any and all publicly available documents described herein, including any and all cited U.S. patents, patent applications, and non-patent publications, are specifically incorporated by reference herein in their entirety. Nonetheless, the related art and publications described herein are not intended in any way as an admission that any of the documents described therein, including pending U.S. patent applications, are prior art to embodiments of the present disclosure. Moreover, the description herein of any disadvantages associated with the described products, methods, and/or apparatus, is not intended to limit the disclosed embodiments. Indeed, embodiments of the present disclosure may include certain features of the described products, methods, and/or apparatus without suffering from their described disadvantages.

Naturally, further objects of the invention are disclosed throughout other areas of the specification, drawings, and claims.

### BRIEF DESCRIPTION OF THE DRAWINGS

FIG. 1 is an illustration of the pelvic girdle showing a needle advanced through the iliac crest and into bone marrow to aspirate bone marrow from the ilium.

FIG. 2A-F are photo micrographs of prepared BMC in culture as prepared for one embodiment of the novel technology.

FIG. 3A-B are graphical representations of CFU-O and Cell Phenotype, respectively, versus CFU-F frequency in BMC samples as prepared for one embodiment of the novel technology.

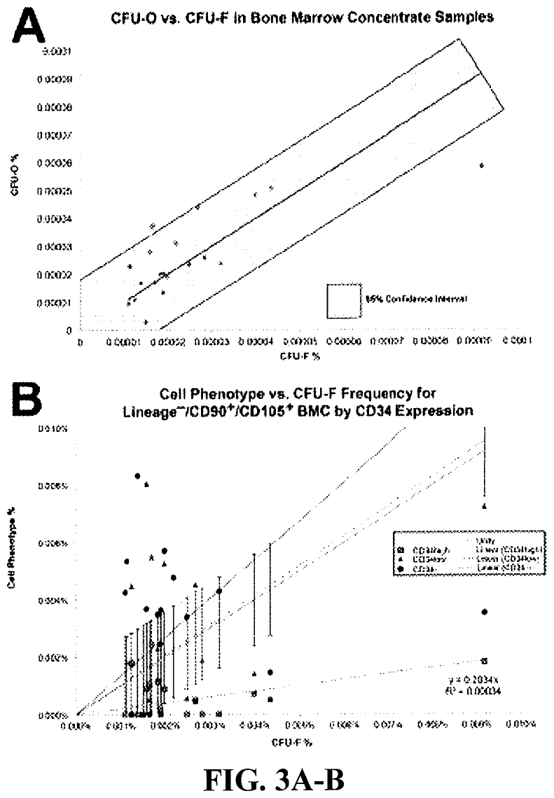

FIG. 4 is a graphic that shows bone marrow aspiration from the ilium.

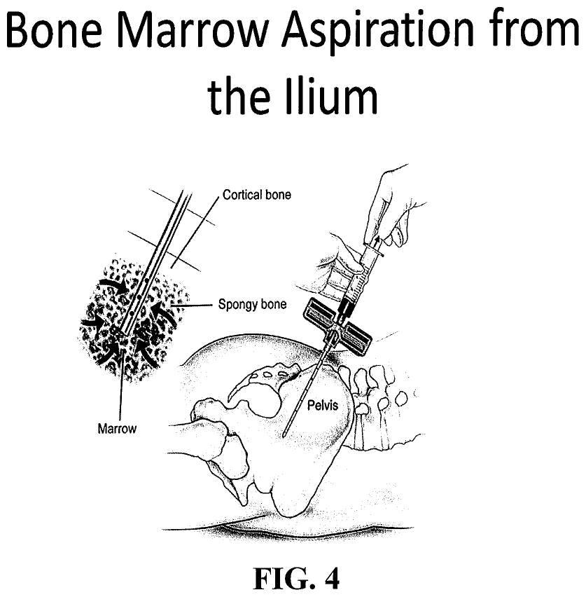

FIG. 5 is a diagram that shows a Jamshidi bone biopsy needle and apparatus to aspirate bone marrow, including passage through a cancellous bone plug.

FIG. 6 is a copy of published tabular data (from Pettine, K. A., et al., “Percutaneous Injection of Autologous Bone Marrow Concentrate Cells Significantly Reduces Lumbar Discogenic Pain Through 12 Months,” Stem Cells 2015, 33:146-156), which shows what the cell counts are with the traditional method of bone marrow aspiration, in contrast to the novel technique described herein of aspirating through a cancellous bone plug, resulting in substantial increase in cell counts.

### DETAILED DESCRIPTION OF THE INVENTION

Before the present methods, implementations and systems are disclosed and described, it is to be understood that this invention is not limited to specific components, specific methods, specific implementation, or to particular compositions, and as such may, of course, vary. It is also to be understood that the terminology used herein is for the purpose of describing particular implementations only and is not intended to be limiting. Neither are mechanisms which have been provided to assist in understanding the disclosure meant to be limiting.

The present invention includes MSC-containing compositions that are useful in treating autoimmune conditions and promoting rapid healing from acute traumatic soft tissue orthopedic injuries. According to one embodiment, such a composition is achieved by incorporating a population of fresh or expanded MSC's, or extracts or derivatives thereof. According to another embodiment, such a composition is achieved by incorporating BMC that includes a population of fresh or expanded MSCs.

For the purposes of this disclosure, MSCs may be derived from multiple sources including, bone marrow stroma, adipose, blood, dermis, periosteum and tissues. In various embodiments, MSCs are extracted from a patient intended to receive the cellular therapy (i.e., an autologous transplant). MSCs can be culture-expanded prior to their introduction into the patient. Growth of MSCs in vitro can be used, for example, to increase the number of MSCs available for infusion, implantation or injection. In non-limiting examples, MSC numbers are increased about two-fold or greater, about ten-fold or greater, or about twenty-fold or greater or more, depending on the desired number of cells. Patient-specific master cell banks, for example can include ten or more passages of expanded autologous cells, which increases MSC counts by many orders of magnitude in comparison to primary isolates. In various embodiments, growing MSCs in vitro can include expansion in a cell culture medium, which includes nutrients, buffers, salts, proteins, vitamins and/or growth factors, which promote MSC growth. Illustratively, a useful cell culture medium is RPMI 1680 supplemented with 10% serum, and appropriate antibiotics such as penicillin/streptomycin and G418. Human serum is preferred for expanding the MSCs for human transplant preparations, but fetal bovine serum has produced acceptable yields of the MSCs in culture. A currently preferred culture system is an environmentally controlled, closed-culture system, commonly known as a bioreactor, which is particularly useful for collection of MSC-conditioned media. Other systems are suitable for culture of MSCs, the expansion of donor cells, and the creation of master MSC banks. These should comply with Good Tissue Practices and ideally should be cGMP. The expanded cells are commonly but not necessarily frozen prior to implantation or administration, and a suitable cryogenic medium that is acceptable to the FDA for such purposes is Cryo-StorCSIO, available from Bio-Life Solutions, Bothell, Wash.

In one embodiment, the MSC-containing products of the invention are substantially free from non-human animal products. Another embodiment of the invention provides for the use of a medium conditioned by growth of MSCs to prepare a MSC-containing product for IV infusion into a patient. Another embodiment of the invention provides for the use of MSC-derived products from primary, expanded or transgene expressing MSCs to prepare a product for IV-infusion to a subject.

The methods of the invention have wide applicability to the treatments described herein. The mode of administration is typically via IV infusion, but administration and dosage regimens vary depending on the particular condition for which modulation is sought.

As used herein, the term “effective amount” encompasses an amount sufficient to effect beneficial or desired therapy results. An effective amount is administered in one or more administrations. An “effective amount” may be of MSCs and MSC products or a combination thereof used alone or in conjunction with one or more agents used to modulate a condition being treated. The duration of treatment generally will depend on the response of the patient's condition to the treatment.

BMA (bone marrow aspirate) and BMC are known to contain hematopoietic as well as MSC populations. The method of extraction is typically correlative to the source of these cells. Typically, the novel technology utilizes an approach, whereby approximately 10 cc of BMA is drawn with typically frequent rotation and repositioning. Additional draws are done after deeper placement of a needle in the iliac crest.

Bone marrow collection and processing to BMC may be carried out herein as described in Pettine, K. A., U.S. Pat. No. 9,408,874 (Aug. 9, 2016), which is incorporated herein by reference in its entirety. This provides a pre-determined amount of a processed BMC with a pre-determined amount of a pre-mixture, where the pre-mixture includes quantities of anticoagulant solution, dextrose and phosphate buffered saline. Typically, the iliac crest, and more typically the posterior iliac crest, is where bone marrow aspirate may be harvested in a surgical setting; however, any suitable area where BMA may be extracted may be used. The novel cellular “snapshot” is derived via timely methodologies specific to non-expanded, minimally manipulated, autologous cell and associated endogenous microenvironment (“milieu”). Typically, once extracted, the BMA and the resulting BMC is obtained by isolation of the desired cell populations via centrifugation. However, any suitable means known in the art may be used to obtain the BMC from the BMA. When centrifugation is used, the BMA is separated according to the slightly differing specific gravities of the aspirated cell types. The cells contained in the BMA can be stratified under centrifugation. The volume, rate, and time of centrifugation are important for controlling the resulting biologic factors contained within the endogenous milieu. Typically, the longer the processing time and/or the more agitation and handling, the lower the oxygen level in the extracted cells which typically include cellular constituents and components sequestered in the residual endogenous milieu, wherein the milieu typically includes antigens, surface biomarkers, proteins and growth factors for angiogenesis, osteogenesis, other regenerative outcomes, and the like. In some embodiments, the degradative manipulation and resultant influence during processing is limited. The resulting, unadulterated milieu may retain a large number of unchanged biologic drivers, markers and signals that are dose appropriate and specific to the cascade of healing found through the native physiology. The stratification and selection of MSCs and progenitors from this population may influence traits such as specific plasticity and immunomodulation. Typically, the time between extraction and re-implantation, re-injection, or IV infusion is within 1 hour, and more typically within 30 minutes, and still more typically within 20 minutes; but, longer time periods may be used if needed. Thus, a point of care approach may be typically implemented with the novel technology. Optionally, once the BMA has been centrifuged, the resulting stratified cell layers may be prepared for delivery to the patient. Typically, the containers, anticoagulants used, and the delivery media used for interim storage and delivery are also prepared during this step. In some embodiments, preserving endogenous proteins, structure and morphology resides within this step, as too great a deviation from the oxygen, microenvironment and stress factors can lead to changes in the composition of the milieu. Typically, the proteins, structure, and morphology are not significantly altered. In one embodiment, the novel delivery media formulation is tailored to preserve the extracted cells and their endogenous factors, while maintaining cell health and identity. Typically, a premixture including an aqueous solution of anticoagulant (ACD-A), an equal amount of dextrose (50%), and phosphate buffered saline (PBS), or the like is pre-mixed and aliquoted in a volume to typically match or approximate the cellular matrix extracted from the centrifugation stratification layers at a ratio of about 1:1. More typically, the premixture is added to the cellular matrix with specific volumes being matched to, or slightly greater than 50/50 by volume, although the ratio may be greater, such as 2:1 or even higher. In some embodiments, the steps in extracting, isolating, separating, re-extracting, dosing, mixing, and delivery impact the cell population, endogenous proteins, surface structural and biomarkers, associated with the compositional regenerative capacity. Typically, the shorter the time consumed by the above-mentioned steps, the less adulterated the original milieu composition will become.

In one embodiment, provided herein is a method of treatment of autoimmune conditions, promoting rapid healing from acute traumatic soft tissue orthopedic injuries, and treating alcohol related liver pathology, and the like, said method comprising IV infusion of the MSC-containing and/or the BMC-containing products of the invention. In one key aspect of the method, the MSC-containing products and/or the BMC-containing products are subjected to a step of bathing or mixing with a glucose-containing sauce for a period of time, prior to the step of infusing them intravenously into a patient. Illustratively, the bathing or mixing may be done by stirring or agitation or any suitable mixing technique known in the art. The glucose-containing sauce comprises a glucose solution having a glucose concentration of from about 25% to about 75% by volume, preferably of from about 40% to about 60% by volume, and most preferably of about 50% by volume. The glucose solution includes glucose dissolved (or suspended or emulsified) in a suitable medium, preferably in an aqueous medium, or in a mixture of an aqueous medium and one or more other suitable media; examples of the one or more other suitable media include any of the art-recognized media used in intravenous treatments. The bathing period of time may vary from about 1 minute to about 30 minutes, or longer if needed. Thus, illustratively, the bathing period of time may be between about 1 minute to about 5 minutes, between about 5 minutes and about 10 minutes, between about 10 minutes and about 15 minutes, between about 15 minutes and about 20 minutes, between about 20 minutes and about 25 minutes, between about 25 minutes and about 30 minutes, or longer than 30 minutes if needed. Further details of the glucose-bathing step are described in the Examples section below.

Glucose is known to be the primary energy source for most cells of the body. Glucose metabolism is central to cell proliferation, growth, and survival. Without being bound by theory, it is believed that bathing the MSCs with glucose results in the glucose being transported across the plasma membrane of the MSC by a facilitative glucose transporter within the intracellular matrix. Hexokinase on the mitochondria then phosphorylates glucose to glucose 6-phosphate. The glucose 6-phosphate enters the glycolytic pathway, generating NADH, ATP and pyruvate. The main function of ATP is to maximize mitochondrial function. The goal of bathing the BMC in glucose is to maximize the cells levels of ATP to maximize their mitochondrial function prior to IV infusion.

Another embodiment of the methods of the invention pertains to filtering of the MSCs or MSC-containing BMC to remove any bone debris or non-cellular contamination found in the MSCs (or BMC), prior to treatment via IV infusion. The filter size is very important. The average size of a MSC is 10 to 12 μm, up to 18 μm. The size of a red blood cell is 6 to 8 μm in a platelet. Accordingly, filtration of the MSCs (or BMC) through an 18 μm filter is chosen to maximize the concentration of MSCs by taking out anything larger than 18 μm.

Another embodiment of the invention provides one or more novel MSC and/or BMC therapeutic products and associated novel methods of obtaining them from a patient and of using them in therapeutic treatments of the patient via IV infusion. In one key aspect, the MSC therapeutic products comprise MSC-containing autogenous BMC that has not been subjected to expansion in a laboratory, and wherein the BMC includes many cell types and the cells are directly from the patient. In contrast, all literature publications to-date discussing IV infusion of MSCs pertain to cells that have been expanded in a laboratory and are of pure MSC infusions, and none discuss IV infusion of BMC that includes many cell types that are directly from the patient and that have not been expanded in a laboratory.

Another embodiment of the invention provides a method that comprises a novel technique for obtaining the MSCs and/or MSC-containing BMC therapeutic products from a patient, wherein the novel technique results in maximizing cell counts in said therapeutic products. In one aspect, the novel technique allows for aspirating bone marrow from the patient in a manner that, surprisingly and unexpectedly, increases the cell count by as much as 10 times, when compared to cell counts obtained via the traditional method of bone marrow aspiration. This technique involves passing the bone marrow aspirate through one or more plugs of cancellous bone, as is described in the Examples section below.

Treatment of Fibromyalgia.

In one embodiment of the invention, the therapeutic MSC products and associated methods of use disclosed herein provide a treatment for fibromyalgia, as described below in the Examples section. Fibromyalgia affects approximately 5,000,000 people in the United States. Between 80 and 90% of those diagnosed with fibromyalgia are women. This is a chronic disorder characterized by hypersensitivity to pain resulting in diffuse tenderness, fatigue, memory abnormalities, sleep disturbances, headaches, and can also result in hypersensitivity to temperature, noises and bright light. There is no specific blood test to diagnose this chronic debilitating condition. The American College of Rheumatology required a history of widespread pain lasting more than three months and other general physical symptoms including fatigue, awaking unrefreshed and memory or thought abnormalities to make the diagnosis. Often, a physician will order various blood tests to rule out other specific rheumatologic conditions that may mimic fibromyalgia. Most experts in the field consider fibromyalgia to be a rheumatic condition. Other conditions such as rheumatoid arthritis or lupus are associated with the development of fibromyalgia. These are all autoimmune disorders. Only three medications, duloxetine (Cymbalta), milnacipran (Savella) and pregabalin (Lyrica) are approved by the US FDA for the treatment of fibromyalgia. Cymbalta was originally developed for and is still used to treat depression. Savella is similar to a drug used to treat depression, but is FDA approved only for fibromyalgia. Lyrica is a medication developed to treat neuropathic pain. Many patients with a diagnosis of fibromyalgia are treated with a cornucopia of pharmacologic agents including analgesics, such as nonsteroidal anti-inflammatory medications and even narcotics. Various sleep medications are often prescribed including Ambien. Patients with fibromyalgia may find themselves also taking various anti-depressants, Neurontin, low dose naltrexone, all of which would be FDA off label uses of these medications when prescribed for fibromyalgia. Most all experts in the field recommend the following: exercising (even though this may be extremely painful), maximizing a healthy diet, and reducing stress in your life. All types of alternative medicine techniques have been utilized for the treatment of fibromyalgia. Some of these include warm water therapy, acupuncture, massage therapy, yoga, Tai chi, Pilates, as well as chiropractic care and physical therapy. Non-prescription medications of all sorts have been also utilized including various herbs and dietary supplements.

Further relevant information on fibromyalgia may be found in the following publications, which are incorporated herein by reference in their entirety:
Fibromyalgia. National Institute of Arthritis and Musculoskeletal and Skin Diseases. http://www.niams.nih.gov/Health Info/Fibromyal agia/default.asp. Accessed Aug. 19, 2013
Clauw D J, et al. The science of fibromyalgia. Mayo Clinic Proceedings, 2011, 86:907.
Arnold L M, et al. A framework for fibromyalgia management for primary care providers. Mayo Clinic Proceedings. 2012, 87:488.
Goldenberg D L. Pathogenesis of fibromyalgia. http://www.uptodate.com/home. Accessed Aug. 19, 2013.
Goldenberg D L. Treatment of fibromyalgia in adults not responsive to initial therapies. http://www.uptodate.com/home. Accessed Aug. 19, 2013.
Crofford L J. Adverse effects of chronic opiod therapy for chronic musculoskeletal pain. Nature Reviews Rheumatology. 2010, 6:191.
Jso Y, et al. Multipotent human stromal cells improve cardiac function after myocardial infarction in mice without long term engraftment. Biochem Biophys Res Commun 2007, 354:700-706 [Pubmed: 17257581].
Lee R H, et al. Intravenous hMSCs improve myocardial infarction in mice because cells embolized in lungs are activated to secrete anti-inflammatory protein TSG-6. Cell Stem Cell 2009, 5:54-63. [Pubmed: 19570514].
Ortiz L A, et al. Interleukin1 receptor antagonist mediates the anti-inflammatory and antifibrotic effect of mesenchymal stem cells during lung injury. Proc Nat Acad Sci U.S.A. 2007, 104:11002-11007 [Pubmed: 17569781].
Togel F, et al. Vasculotropic paracrine actions of infused mesenchymal stem cells are important to the recovery from acute kidney injury. Am J Physiol Renal Physiol 2007, 292:F1626-F1635 [Pubmed 17213465].
Ankrum J, Karp J M. Mesen chymal stem cell therapy: Two steps forward, one step back. Trends Mol Med. 2010, 16(5):203-209. Doi:10.1016/j.molmed.2010.02.005.

Treatment of Multiple Sclerosis.

In one embodiment of the invention, the therapeutic MSC products and associated methods of use disclosed herein provide a treatment for multiple sclerosis, as described below in the Examples section. Multiple Sclerosis (MS) is a disease in which a patient's immune system attacks the protective sheath (Myelin) that covers the patient's nerves. Myelin can be compared to the insulation on electric wires. When myelin is damaged, the message that travels along that nerve may be slowed, blocked or interfered with by nearby nerves. Myelin damage disrupts communication between the brain and the rest of the body. Ultimately, the nerves themselves may deteriorate, a process that is currently irreversible. Signs and symptoms vary widely, depending on the amount of damage and which nerves are affected. Some people with severe MS may lose the ability to walk independently or at all, while others experience long periods of remission, during which they develop no new symptoms. There is no cure for multiple sclerosis, however, treatments can help speed recovery from attacks, modify the course of the disease and manage symptoms. Signs and symptoms of MS vary, depending on the location of affected nerve fibers. MS signs and symptoms may include the following: Numbness or weakness in one or more limbs, partial or complete loss of vision, tingling or pain in parts of one's body, electric shock sensations, tremor, lack of coordination or steady gait, slurred speech, fatigue, dizziness, problems with bowel and bladder function. The cause of MS is unknown. It is believed to be an auto-immune disease similar to Crohn's disease, Rheumatoid Arthritis, and Lupus. There are several types of MS. The most common form is relapsing-remitting MS. This type of MS is characterized by attacks or relapses that can develop over days or weeks and then improve followed by a period of very few symptoms. Although symptoms generally resolve after a relapse, symptoms still gradually progress after each relapse and in about 60-70% of cases it becomes another type called secondary progressive MS where the relapses stop but symptoms begin to steadily get worse. A third type of MS is a gradual onset and steady progression of signs and symptoms with no relapses. This is called primary-progressive MS. The fourth type is a combination of primary progressive and relapse remitting, and progresses the fastest. It is called progressive relapsing MS and is characterized by relapses where the symptoms do not return to baseline, followed by gradual increase of symptoms between relapses.

The demographics of MS include the following: It most commonly affects people between the ages of 15 and 60 with women about twice as likely as men to develop MS. There is a genetic component in that a family member who has MS increases one's risk of developing this autoimmune disease. People of Northern European descent are at a higher risk, which correlates with the general trend of those living further from the equator having a higher risk.

The diagnosis of multiple sclerosis is typically based on a number of tests, symptoms and exclusion of other diseases. These include an MRI scan of one's brain, which has characteristic white lesions, separate incidences of relapse, and testing for inflammatory markers. A spinal tap to remove cerebral spinal fluid can show abnormalities in one's white blood cells or antibodies that are associated with MS called oligo-clonal banding.

No current treatment for MS results in a cure. All treatments used are only to help reduce the symptoms, slow progression, and reduce relapses. The mainstay of treatment for acute relapse is steroids, which may include oral prednisone or intravenous methylprednisone. There are many side effects to the use of steroids, especially if they are used longer than a few days. If symptoms are severe and have not responded to steroids, plasmaphoresis can be utilized. This involves removing the plasma from one's blood and then mixing the red blood cells with a protein solution (albumin). The mixture is then placed back into one's vein. The goal of plasmaphoresis is to remove the harmful inflammatory proteins/chemicals from one's blood plasma while keeping one's red blood cells and blood volume intact.

No treatments have shown benefit for slowing the progression of primary-progressive MS. Treatments for relapsing-remitting MS may lower the relapse rate and reduce the rate of new lesion formation. These treatments include beta interferons (Avonex, etc.), which are injected under the skin or into muscle. Copaxone (glatiramer acetate) is a medication to block one's immune system's attack on one's myelin sheath by mimicking the myelin components and distracting the immune system. This medication is injected beneath the skin. Tecfidera (dimethyl fumarate) is an oral medication, in an attempt to reduce relapses. Gilenya (fingolimod) is an oral medication to also help reduce relapses by trapping immune cells in lymph nodes where they can't attack the brain. Aubagio (teriflunomide) is an oral medication similar to many chemotherapy drugs also used to help reduce the occurrence of relapses. Tysabri (natalizumab) is an oral medication designed to block immune cells traveling from your blood stream to your brain. Novantrone (mitoxantrone) is an immunosuppressant chemotherapy drug to help reduce symptoms, but is usually only used to treat very severe, advanced MS (including secondary progressive MS). All of these medications have side effects including liver damage, increased blood pressure, blurred vision, nausea, diarrhea, lowered white blood count with increased risk of infections. Many also can (rarely) cause a serious infectious brain disease called progressive multifocal leukoencephalopathy, which is similar to MS but rapidly progressive and almost always fatal. Most physicians agree that living with MS is improved with exercise, eating a balanced diet, obtaining optimal amounts of rest and relieving stress.

Further relevant information on multiple sclerosis may be found in the following publications, which are incorporated herein by reference in their entirety:
Mayo Clinic—Diseases and Conditions-Multiple sclerosis. Mayoclinic.org Accessed Sep. 14, 2015.
Olek M J. Epidemiology and clinical features of multiple sclerosis in adults. http://uptodate.com/home. Accessed Apr. 4 2014.
Olek M J. Treatment of relapsing-remitting multiple sclerosis in adults. http://uptodate.com/home. Accessed Apr. 4, 2014.
Olek M J. Treatment of acute exacerbations of multiple sclerosis in adults. http://uptodate.com/home. Accessed Apr. 4, 2014.
Wingerchuck O M. Multiple Sclerosis: Current and emerging disease-modifying therapies and treatment strategies. Mayo Clinic Proceedings. 2014, 89:225.
Pizzorno J E, et al. Textbook of Natural Medicine. 4th ed. St. Louis, Mo.: Churchill Livingstone Elsevier; 2013. https://www.clinicalkey.com Accessed Apr. 4, 2014.
Kantarci 0. Treatment of Primary Progressive Multiple Sclerosis. Seminars in Neurology. 2013, 33:74.
Iso Y, et al. Multipotent human stromal cells improve cardiac function after myocardial infarction in mice without long term engraftment. Biochem Biophys Res Commun. 2007, 354:700-706 [Pubmed: 17257581].
Lee R H, et al. Intravenous hMSCs improve myocardial infarction in mice because cells embolized in lungs are activated to secrete anti-inflammatory protein TSG-6. Cell Stem Cell 2009, 5:54-63 [Pubmed: 19570514].
Ortiz L A et al. Interleukin1 receptor antagonist mediates anti-inflammatory and anti-fibrotic effect of mesenchymal stem cells during lung injury. Proc Natl Acad Sci U.S.A. 2007, 104:1102-1107 [Pubmed: 17569781].
Togel F, et al. Vasculotropic paracrine actions of infused mesenchymal stem cells are important to the recovery from acute kidney injury. Am J Physiol Renal Phsyiol. 2007, 292:F1626-F1635.
Onken J, Gallup D, Hanson J, Pandak M, Custer L. Successful treatment of refractory Crohn's Disease using Adult Mesenchymal Stem Cells. (October 2006).
Loftus J R E V. Clincal epidemiology of inflammatory bowel disease: incidence, prevalence, and environmental influences. Gastroenterology. 2004, 126(6):1504-17.
Parachette K R, Makam R C, Cuffari C. Infliximab therapy in pediatric Crohn's disease: a review. Clin Exp Gastroenterol. 2010, 3:57-63.
Ciccocioppo R et al. Autologous bone marrow-derived mesenchymal stromal cells in the treatment of Crohn's disease. Gut. 2011, 60(6):788-798.
Garcia-Bosch O, Ricart E, Panes J. Review article: stem cell therapies for inflammatory bowel disease-efficacy and safety. Aliment Pharmacal Ther 2010, 32(8):939-52.
Lazarus H M et al. Ex Vivo expansion and subsequent infusion of human bone marrow-derived stromal progenitor cells (mesenchymal progenitor cells): implications for therapeutic use. Bone Marrow Transplant. 1995, 57(1):11-20.
Cohen J A. Mesenchymal stem cell transplantation in multiple sclerosis. Journal of the Neurological Sciences 333 (2013) 43-49.
Ankrum J, Karp J M. Mesenchymal stem cell therapy: Two steps forward, one step back. Trends Mol Med. 2010 May, 16(5):203-209. Doi: 10.1016/j.molmed.2010.02.005.
Glenn, J D, Whartenby K A. Mesenchymal stem cells: emerging mechanisms of immunomodulation and therapy. World J Stem Cells 2014 26, 6(5):526-539.
Katata M, Dezawa M. Parkinson's Disease and mesenchymal stem cells: Potential for cell based therapy. Parkinson's Disease 2012, Article ID 873706, 9 pages. doi: 10.1155/2012.873706.
Llufriu S, et al. Randomized placebo-controlled phase 2 trial of autogenous mesenchymal stem cells in Multiple Sclerosis. PLoS ONE 9(12): e113936.doi:10.1371/journal.pone.0113936.
Uccelli A, Laroni A, Freedman M S (2011) Mesenchymal stem cells for the treatment of multiple sclerosis and other neurological diseases. Lancet Neurol 10:649-656.
Rice C M, Kemp K, Wilkins A, Scolding N J (2013) Cell therapy for multiple sclerosis: an evolving concept with implications for other neurodegenerative diseases. Lancet 382:1204-1213.
Yamout B, et al. Bone marrow mesenchymal stem cell transplantation in patients with multiplesclerosis: A pilot study. Journal of Neuroimmunology 227 (2010) 185-189.
Karussis D, et al. Safety and Immunological Effects of Mesenchymal Stem Cell Transplantation in Patients With Multiple Sclerosis and Amyotrophic Lateral Sclerosis. Arch Neural. 2010 October; 67(10):1187-1194.doi:10.1001/archneurol.2010.248.
Horwitz E M, Prockop D J, Fitzpatrick L A, et al. Transplantability and therapeutic effects of bone marrow-derived mesenchymal cells in children with osteogenesis imperfecta. Nat Med 1999, 5(3):309-313. [PubMed: 10086387].
Horwitz E M, Prockop D J, Gordon P L, et al. Clinical responses to bone marrow transplantation in children with severe osteogenesis imperfecta. Blood 2001, 97(5):1227-1231. [PubMed: 11222364] Karussis et al. Page 9.
Wollert K C, Meyer G P, Lotz J, et al. Intracoronary autologous bone-marrow cell transfer after myocardial infarction: the BOOST randomised controlled clinical trial. Lancet 2004, 364(9429):141-148. [PubMed: 15246726].
Stamm C, Westphal B, Kleine H D, et al. Autologous bone-marrow stem-cell transplantation for myocardial regeneration. Lancet 2003, 361(9351):45-46. [PubMed: 12517467].
Perin E C, Dohmann H F, Borojevic R, et al. Transendocardial, autologous bone marrow cell transplantation for severe, chronic ischemic heart failure. Circulation 2003, 107(18):2294-2302. [PubMed: 12707230].
Perin E C, Dohmann H F, Borojevic R, et al. Improved exercise capacity and ischemia 6 and 12 months after transendocardial injection of autologous bone marrow mononuclear cells for ischemic cardiomyopathy. Circulation 2004, 110(11 suppl 1):11213-11218. [PubMed: 15364865].
Kor; O N, Day J, Nieder M, Gerson S L, Lazarus H M, Krivit W. Allogeneic mesenchymal stem cell infusion for treatment of metachromatic leukodystrophy (MLD) and Hurler syndrome (MPS-IH). Bone Marrow Transplant 2002, 30(4):215-222. [PubMed: 12203137].
Assmus B, Schachinger V, Teupe C, et al. Transplantation of progenitor cells and regeneration enhancement in acute myocardial infarction (TOPCARE-AMI). Circulation 2002, 106(24):3009-3017. [PubMed: 12473544].
Chen S L, Fang W W, Ye F, et al. Effect on left ventricular function of intracoronary transplantation of autologous bone marrow mesenchymal stem cell in patients with acute myocardial infarction. Am J Cardiol 2004, 94(1):92-95. [PubMed: 15219514].
Ferrari G, Cusella-De Angelis G, Coletta M, et al. Muscle regeneration by bone marrow-derived myogenic progenitors [published correction appears in Science. 1998, 281(5379):923). Science 1998, 279(5356):1528-1530. [PubMed: 9488650].
Katritsis D G, Sotiropoulou P A, Karvouni E, et al. Transcoronary transplantation of autologous mesenchymal stem cells and endothelial progenitors into infarcted human myocardium. Catheter Cardiovasc Interv 2005, 65(3):321-329. [PubMed:15954106].
Kor; O N, Gerson S L, Cooper B W, et al. Rapid hematopoietic recovery after coin-fusion of autologous-blood stem cells and culture-expanded marrow mesenchymal stem cells in advanced breast cancer patients receiving high-dose chemotherapy. J Clin Oncol 2000, 18(2):307-316. [PubMed:10637244].
Martino G, Franklin R J, Van Evercooren A B, Kerr D A. Stem Cells in Multiple Sclerosis (STEMS) Consensus Group. Stem cell transplantation in multiple sclerosis: current status and future prospects. Nat Rev Neurol 2010, 6(5):247-255. [PubMed:20404843].
Freedman M S, Bar-Or A, Atkins Hl, et al. MSCT Study Group. The therapeutic potential of mesenchymal stem cell transplantation as a treatment for multiple sclerosis: consensus report of the International MSCT Study Group. Mult Scler 2010, 16(4):503-510. [PubMed:20086020].

Treatment of Parkinson's Disease.

In one embodiment of the invention, the therapeutic MSC products and associated methods of use disclosed herein provide a treatment for Parkinson's Diseases, as described below in the Examples section. Parkinson's disease is a progressive disorder of the nervous system that affects movement. Parkinsonism is characterized by a loss of dopaminergic neurons in the midbrain region known as the striatum. It develops gradually, sometimes starting with a slight tremor in just one hand. The disorder also commonly causes stiffness or slowing of movement. In the early stages of Parkinson's Disease, the patient may notice a decrease in facial expressions or the fact that one's arms do not swing while walking. One's speech may become soft or slurred. Parkinson's Disease symptoms worsen as one's condition progresses over time. Symptoms vary from person to person. Parkinson's Disease usually begins at the age of 60 or older. Men are more likely to develop Parkinson's Disease than women and there appears to be a hereditary component. Other symptoms may include difficulty in thinking processes, depression, emotional changes, swallowing problems, sleep disorders, and bladder difficulties. There is no cure for Parkinson's Disease. Medications can be utilized in an attempt to decrease one's symptoms. The most common medication used to treat Parkinson's is levodopa, a compound that is metabolized in the brain into dopamine. Levodopa can be combined with carbidopa to increase dopamine concentrations in the brain. These medications include Rytary and Sinemet. As Parkinson's Disease progresses, the benefits from levodopa tend to decrease, and the medication can begin to cause involuntary spastic movements called dyskinesias. The FDA approved a drug called Duopa in 2015, which is levodopa, placed through a feeding tube directly into the small intestine. Other drugs have been developed to mimic the dopamine effects in the patient's brain. They generally are not as effective as levodopa, but these include Mirapex, Requip, Apokyn, and a skin patch medication called Meupro. All of these medications have side effects, such as hallucinations, compulsive behaviors and sleepiness. Other medications have been developed to prevent the breakdown of brain dopamine called MA0-8 inhibitors. Finally, medications, called Catechol-O-methyltransferase inhibitors have been developed to help prevent the breakdown of dopamine, prolonging the effects of levodopa. The tremors associated with Parkinson's Disease have been treated with anticholinergics. These medications have side effects including impaired memory, confusion, hallucinations, dry mouth, and impaired urination. Surgical procedures to treat Parkinson's Disease include deep brain stimulation where electrodes are implanted in specific parts of the brain. These are connected to a generator implanted near the chest that sends electrical pulses to the patient's brain and may reduce symptoms of Parkinson's Disease. General habits can help treat Parkinson's Disease, and include living a healthy lifestyle. This includes exercise, healthy eating, and attempting to avoid falls. Other alternative medicine treatments that may help the symptoms of Parkinson's Disease include the use of Coenzyme Q10, massage, acupuncture, Tai Chi, yoga, Alexander technique, meditation, music or art therapy and pet therapy.

Further relevant information on Parkinson's Disease may be found in the following publications, which are incorporated herein by reference in their entirety:
Mayo Clinic Staff. Mayo Clinic: Diseases and Conditions—Parkinson's disease. http://mayoclinic.org/diseases-conditions/parkinsons-disease. Accessed Sep. 14, 2015.
Glavaski-Joksimovic A, Bohn M C. Mesenchymal stem cells and neuro regeneration in Parkinson's disease. Experimental Neurology (2013) 247: 25-38.
Longo D L et al. Parkinson's disease and other movement disorders. In: Harrison's Principles of Internal Medicine. 18th ed. New York, N.Y.: The McGraw-Hill Companies; 2012. http://accessmedicine.com. Accessed Apr. 6, 2015.
Chou K L. Diagnosis of Parkinson's disease. http://www.uptodate.come/home.
Tarsy D., Pharmacologic treatment of Parkinson's disease. http://www.uptodate.com/home.
Parkinson's: Fitness Counts National Parkinson Foundation. http://parkinson.org/searchpages/search.aspx?pKeywords=fitness. Accessed Apr. 9, 2015.
Parkinson's disease. Natural Medicines Comprehensive Database. http://www.naturaldatabase.com. Accessed Apr. 6, 2015.
Tarsy D. Nonpharmacologic management of Parkinson disease. http://www.uptodate.com/home/. Accessed Apr. 6, 2015.
Complementary therapies and Parkinson's. Parkinson's Disease Society of the United Kingdom. http://www.parkinsons.org.uk/content/complementary-therapies-and-Parkinsons-booklet. Accessed Apr. 9, 2015.
Abbvie announces U.S. approval of Duopa (carbidopa and levodopa) enteral suspension for the treatment of motor fluctuations in patients with advanced Parkinson's disease. http:/labbvie.mediaroom.com/2015-01-12-Abbvie-Announces-U—S-FDA-Approval-of-DUOPA-carbidopa-levodopa-enteral-suspension-for-the-treatment-of-motor-fluctuations-in-patients-with-advanced-Parkinsons. Accessed Apr. 20, 02105.
Iso Y, et al. Multipotent human stromal cells improve cardiac function after myocardial infarction in mice without long term engraftment. Biochem Biophys Res Commun. 2007, 354:700-706 [Pubmed: 17257581].
Lee R H, et al. Intravenous hMSCs improve myocardial infarction in mice because cells embolized in lungs are activated to secrete anti-inflammatory protein TSG-6. Cell Stem Cell 2009, 5:54-63 [Pubmed: 19570514].
Ortiz L A et al. Interleukin1 receptor antagonist mediates anti-inflammatory and anti-fibrotic effect of mesenchymal stem cells during lung injury. Proc Natl Acad Sci U.S.A. 2007, 104:1102-1107 [Pubmed: 17569781].
Togel F, et al. Vasculotropic paracrine actions of infused mesenchymal stem cells are important to the recovery from acute kidney injury. Am J Physiol Renal Phsyiol. 2007, 292:F1626-F1635.
Ciccocioppo R et al. Autologous bone marrow-derived mesenchymal stromal cells in the treatment of fistulising Crohn's disease. Gut. 2011, 60(6):788-798.
Garcia-Bosch O, Ricart E, Panes J. Review article: stem cell therapies for inflammatory bowel disease-efficacy and safety. Aliment Pharmacal Ther. 2010, 32(8):939-52.
Lazarus H M et al. Ex Vivo expansion and subsequent infusion of human bone marrow-derived stromal progenitor cells (mesenchymal progenitor cells): implications for therapeutic use. Bone Marrow Transplant. 1995, 57(1):11-20.
Glenn, J D, Whartenby K A. Mesenchymal stem cells emerging mechanisms of immunomodulation and therapy. World J Stem Cells 2014, 26:6(5):526-539.
Katata M, Dezawa M. Parkinson's Disease and mesenchymal stem cells: Potential for cell based therapy. Parkinson's Disease 2012, Article ID 873706, 9 pages doi: 10.1155/2012.873706.
Blandini F, Cava L, et al. Transplantation of undifferentiated human mesenchymal stem cells protects against 6-hydroxydopamine neurotoxicityin the rat. Cell Transplant (2010) 19:203-217.
Bouchez G, et al. Partial recovery of dopaminergic pathway after graft of adult mesenchymal stem cells in a rat model of Parkinson's disease. Neurochem. Int. (2008) 52:1332-1342.
Cho, K. J., Trzaska, K. A., Greco, S. J., McArdle, J., Wang, F. S., Ye, J. H., Rameshwar, P., 2005. Neurons derived from human mesenchymal stem cells show synaptic transmission and can be induced to produce the neurotransmitter substance P by interleukin-1alpha. Stem Cells 23, 383-391.
Cava, L., Armentero, M. T., Zennaro, E., Calzarossa, C., Bossolasco, P., Busca, G., Lambertenghi Deliliers, G., Polli, E., Nappi, G., Silani, V., Blandini, F., 2010. Multiple neurogenic and neurorescue effects of human mesenchymal stem cell after transplantation in an experimental model of Parkinson's disease. Brain Res. 1311, 12-27.
Yang, M., Donaldson, A. E., Marshall, C. E., Shen, J., Iacovitti, L., 2004. Studies on the differentiation of dopaminergic traits in human neural progenitor cells in vitro and in vivo. Cell Transplant. 13, 535-547.
Kim, J. H., Auerbach, J. M., Rodriguez-Gomez, J. A., Velasco, I., Gavin, D., Lumelsky, N., Lee, S. H., Nguyen, J., Sanchez-Pernaute, R., Bankiewicz, K., McKay, R., 2002b. Dopamine neurons derived from embryonic stem cells function in an animal model of Parkinson's disease. Nature 418, SD-56.
Park, H. W., Cho, J. S., Park, C. K., Jung, S. J., Park, C. H., Lee, S. J., Oh, S. B., Park, Y. S., Chang, M. S., 2012. Directed induction of functional motor neuron-like cells from genetically engineered human mesenchymal stem cells. PloS One 7, e35244.
Arnhold, S., Klein, H., Klinz, F. J., Absenger, Y., Schmidt, A., Schinkothe, T., Brixius, K., Kozlowski, J., Desai, B., Bloch, W., Addicks, K., 2006. Human bone marrow stroma cells display certain neural characteristics and integrate in the subventricular compartment after injection into the liquor system. Eur. J. Cell Biol. 85, 551-565.
Chen, X., Katakowski, M., Li, Y., Lu, D., Wang, L., Zhang, L., Chen, J., Xu, Y., Gautam, S., Mahmood, A., Chopp, M., 2002. Human bone marrow stromal cell cultures conditioned by traumatic brain tissue extracts growth factor production. J. Neurosci. Res. 69:687-691.
Crigler, L., Robey, R. C., Asawachaicharn, A., Gaupp, D., Phinney, D. G., 2006. Human mesenchymal stem cell subpopulations express a variety of neuro-regulatory molecules and promote neuronal cell survival and neuritogenesis. Exp. Neural. 198:54-64.
Tate, C. C., Fonck, C., McGrogan, M., Case, C. C., 2010. Human mesenchymal stromal cells and their derivative, SB623 cells rescue neural cells via trophic support following in vitro ischemia. Cell Transplant. 19, 973-984.
Burdon, T. J., Paul, A., Noiseux, N., Prakash, S., Shum-Tim, D., 2011. Bone marrow stem cell derived paracrine factors for regenerative medicine: current perspectives and therapeutic potential. Bone Marrow Res. 2011, 207326.
Kinnaird, T., Stabile, E., Burnett, M. S., Shou, M., Lee, C. W., Barr, S., Fuchs, S., Epstein, S. E., 2004b. Local delivery of marrow-derived stromal cells augments collateral perfusion through paracrine mechanisms. Circulation 109, 1543-1549.
Paul G, Anisimov S V. The secretome of mesenchymal stem cells: Potential implications for neuroregeneration. Biochimie (2013) 95:2246-2256.
Y. S. Fu, Y. C. Cheng, M. Y. Lin, H. Cheng, P. M. Chu, S. C. Chou, Y. H. Shih, M. H. Ko, M. S. Sung, Conversion of human umbilical cord mesenchymal stem cells in Wharton's jelly to dopaminergic neurons in vitro: potential therapeutic application for parkinsonism, Stem Cells 24 (2006) 115e124.
E. J. Kang, Y. H. Lee, M. J. Kim, Y. M. Lee, B. Mohana Kumar, B. G. Jeon, S. A. Ock, H. J. Kim, G. J. Rho, Transplantation of porcine umbilical cord matrix mesenchymal stem cells in a mouse model of Parkinson's disease, Journal of Tissue Engineering and Regenerative Medicine 7(3) (March 2013) 169e182.
Y. S. Levy, M. Bahat-Stroomza, R. Barzilay, A. Burshtein, S. Bulvik, Y. Barhum, H. Panet, E. Melamed, D. Offen, Regenerative effect of neural-induced human mesenchymal stromal cells in rat models of Parkinson's disease, Cytotherapy 10 (2008) 340e352.
M. Li, G. R. Jayandharan, B. Li, C. Ling, W. Ma, A. Srivastava, L. Zhong, High efficiency transduction of fibroblasts and mesenchymal stem cells by tyrosine-mutant AAV2 vectors for their potential use in cellular therapy, Human Gene Therapy 21 (2010) 1527e1543.
D. Offen, Y. Barhum, Y. S. Levy, A. Burshtein, H. Panet, T. Cherlow, E. Melamed, Intrastriatal transplantation of mouse bone marrow-derived stem cells improves motor behavior in a mouse model of Parkinson's disease, Journal of Neural Transmission: Supplementum (2007) 133e143.
P. Shetty, G. Ravindran, S. Sarang, A. M. Thakur, H. S. Rao, C. Viswanathan, Clinical grade mesenchymal stem cells transdifferentiated under xenofree conditions alleviates motor deficiencies in a rat model of Parkinson's disease, Cell Biology International 33 (2009) 830e838.
Y. Wang, J. Yang, H. Li, X. Wang, L. Zhu, M. Fan, X. Wang, Hypoxia promotes dopaminergic differentiation of mesenchymal stem cells and shows benefits for transplantation in a rat model of Parkinson's disease, PLoS One 8 (2013) e54296.
E. F. Wolff, X. B. Gao, K. V. Yao, Z. B. Andrews, H. Du, J. D. Elsworth, H. S. Taylor, Endometrial stem cell transplantation restores dopamine production in a Parkinson's disease model, Journal of Cellular and Molecular Medicine 15 (2011) 747e755.
Danielyan, R. Schafer, A. von Ameln-Mayerhofer, F. Bernhard, S. Verleysdonk, M. Buadze, A. Lourhmati, T. Klopfer, F. Schaumann, B. Schmid, C. Koehle, B. Proksch, R. Weissert, H. M. Reichardt, J. van den Brandt, G. H. Buniatian, M. Schwab, C. H. Gleiter, W. H. Frey 2nd, Therapeutic efficacy of intranasally delivered mesenchymal stem cells in a rat model of Parkinson disease, Rejuvenation Research 14 (2011) 3e16.
R. Somoza, C. Juri, M. Baes, U. Wyneken, F. J. Rubio, Intranigral transplantation of epigenetically induced BDNF-secreting human mesenchymal stem cells: implications for cell-based therapies in Parkinson's disease, Biology of Blood and Marrow Transplantation: Journal of the American Society for Blood and Marrow Transplantation 16 (2010) 1530e1540.
M. Bahat-Stroomza, Y. Barhum, Y. S. Levy, O. Karpov, S. Bulvik, E. Melamed, D. Offen, Induction of adult human bone marrow mesenchymal stromal cells into functional astrocyte-like cells: potential for restorative treatment in Parkinson's disease, Journal of Molecular Neuroscience: MN 39 (2009) 199e210.
L. Cova, M. T. Armentero, E. Zennaro, C. Calzarossa, P. Bossolasco, G. Busca, G. Lambertenghi Deliliers, E. Polli, G. Nappi, V. Silani, F. Blandini, Multiple neurogenic and neurorescue effects of human mesenchymal stem cell after transplantation in an experimental model of Parkinson's disease, Brain Research 1311(2010) 12e27.
Glavaski-Joksimovic, T. Virag, T. A., Mangatu, M. McGrogan, X. S. Wang, M. C. Bohn, Glial cell line-derived neurotrophic factor-secreting genetically modified human bone marrow-derived mesenchymal stem cells promote recovery in a rat model of Parkinson's disease, Journal of Neuroscience Research 88 (2010) 2669e2681.
M. L. Khoo, H. Tao, A. C. Meedeniya, A. Mackay-Sim, D. D. Ma, Transplantation of neuronal-primed human bone marrow mesenchymal stem cells in hemiparkinsonian rodents, PLoS One 6 (2011) e19025.
P. Mathieu, V. Roca, C. Gamba, A. DelPozo, F. Pitossi, Neuroprotective effects of human umbilical cord mesenchymal stromal cells in an immunocompetent animal model of Parkinson's disease, Journal of Neuroimmunology 246 (2012) 43e50.
T. C. Moloney, G. E. Rooney, F. P. Barry, L. Howard, E. Dowd, Potential of rat bone marrow-derived mesenchymal stem cells as vehicles for delivery of neurotrophins to the parkinsonian rat brain, Brain Research 1359 (2010) 33e43.
H. J. Park, J. Y. Shin, B. R. Lee, H. O. Kim, P. H. Lee, Mesenchymal stem cells augment neurogenesis in the subventricular zone and enhance differentiation of neural precursor cells into dopaminergic neurons in the substantia nigra of a parkinsonian model, Cell Transplantation 21 (2012) 1629e1640.
F. Wang, T. Yasuhara, T. Shingo, M. Kameda, N. Tajiri, W. J. Yuan, A. Kondo, T. Kadota, T. Baba, J. T. Tayra, Y. Kikuchi, Y. Miyoshi, I. Date, Intravenous administration of mesenchymal stem cells exerts therapeutic effects on parkinsonian model of rats: focusing on neuroprotective effects of stromal cell-derived factor-1alpha, BMC Neuroscience 11(2010) 52.
T. H. Wang, Z. T. Feng, P. Wei, H. Li, Z. J. Shi, L. Y. Li, Effects of pcDNA3-beta-NGF gene-modified BMSC on the rat model of Parkinson's disease, Journal of Molecular Neuroscience: MN 35 (2008) 161e169.
M. L. Weiss, S. Medicetty, A. R. Bledsoe, R. S. Rachakatla, M. Choi, S. Merchav, Y. Luo, M. S. Rao, G. Velagaleti, D. Troyer, Human umbilical cord matrix stem cells: preliminary characterization and effect of transplantation in a rodent model of Parkinson's disease, Stem Cells 24 (2006) 781e792.
Y. X. Chao, B. P. He, S. S. Tay, Mesenchymal stem cell transplantation attenuates blood brain barrier damage and neuroinflammation and protects dopaminergic neurons against MPTP toxicity in the substantia nigra in a model of Parkinson's disease, Journal of Neuroimmunology 216 (2009) 39e50.
Glavaski-Joksimovic,T. Virag, Q. A. Chang, N. C. West, T. A. Mangatu, M. P. McGrogan, M. Dugich-Djordjevic, M. C. Bohn, Reversal of dopaminergic degeneration in a parkinsonian rat following micrografting of human bone marrow-derived neural progenitors, Cell Transplantation 18 (2009) 801e814.
T. Hayashi, S. Wakao, M. Kitada, T. Ose, H. Watabe, Y. Kuroda, K. Mitsunaga, D. Matsuse, T. Shigemoto, A. Ito, H. Ikeda, H. Fukuyama, H. Onoe, Y. Tabata, M. Dezawa, Autologous mesenchymal stem cell-derived dopaminergic neurons function in parkinsonian macaques, The Journal of Clinical Investigation 123 (2013) 272e284.
M. Inden, K. Takata, K. Nishimura, Y. Kitamura, E. Ashihara, K. Yoshimoto, H. Ariga, O. Honmou, S. Shimohama, Therapeutic effects of human mesenchymal and hematopoietic stem cells on rotenone-treated parkinsonian mice, Journal of Neuroscience Research 91(2013) 62e72.
Y. J. Kim, H. J. Park, G. Lee, O. Y. Bang, Y. H. Aim, E. Joe, H. O. Kim, P. H. Lee, Neuroprotective effects of human mesenchymal stem cells on dopaminergic neurons through anti-inflammatory action, Glia 57 (2009) 13e23.
H. J. Park, P. H. Lee, O. Y. Bang, G. Lee, Y. H. Ahn, Mesenchymal stem cells therapy exerts neuroprotection in a progressive animal model of Parkinson's disease, Journal of Neurochemistry 107 (2008) 141e151.
M. C. Pereira, M. Secco, D. E. Suzuki, L. Janjoppi, C. O. Rodini, L B. Torres, B. H. Araujo, E. A. Cavalheiro, M. Zatz, O. K. Okamoto, Contamination of mesenchymal stem-cells with fibroblasts accelerates neurodegeneration in an experimental model of Parkinson's disease, Stem Cell Reviews 7 (2011) 1006e1017.
O. Sadan, M. Bahat-Stromza, Y. Barhum, Y. S. Levy, A. Pisnevsky, H. Peretz, A. B. Ilan, S. Bulvik, N. Shemesh, D. Krepel, Y. Cohen, E. Melamed, D. Offen, Protective effects of neurotrophic factor-secreting cells in a 6-OHDA rat model of Parkinson disease, Stem Cells and Development 18 (2009) 1179e1190.
P. Shetty, A. M. Thakur, C. Viswanathan, Dopaminergic cells, derived from a high efficiency differentiation protocol from umbilical cord derived mesenchymal stem cells, alleviate symptoms in a Parkinson's disease rodent model, Cell Biology International 37 (2013) 167e180.
D. Shi, G. Chen, L. Lv, L. Li, D. Wei, P. Gu, J. Gao, Y. Miao, W. Hu, The effect of lentivirus-mediated T H and GDNF genetic engineering mesenchymal stem cells on Parkinson's disease rat model, Neurological Sciences: Official Journal of the Italian Neurological Society and of the Italian Society of Clinical Neurophysiology 32 (2011) 41e51.
N. Xiong, Z. Zhang, J. Huang, C. Chen, Z. Zhang, M. Jia, J. Xiong, X. Liu, F. Wang, X. Cao, Z. Liang, S. Sun, Z. Lin, T. Wang, VEGF-expressing human umbilical cord mesenchymal stem cells, an improved therapy strategy for Parkinson's disease, Gene Therapy 18 (2011) 394e402.
Ankrum J, Karp J M. Mesenchymal stem cell therapy: Two steps forward, one step back. Trends Mol Med. 2010 May; 16(5):203-209. Doi: 10.1016/j.molmed.2010.02.005.

Treatment of Crohn's Disease.

In one embodiment of the invention, the therapeutic MSC products and associated methods of use disclosed herein provide a treatment for Crohn's Diseases, as described below in the Examples section. Crohn's disease is an inflammatory bowel disease (IBD). This condition results in inflammation of the lining of the patient's digestive tract. This often leads to severe abdominal pain, diarrhea, fatigue, weight loss, and malnutrition. Currently, there is no cure for Crohn's disease. Various medications can reduce its symptoms and possibly bring about temporary remission. Symptoms of Crohn's disease can range from mild to severe. Symptoms can appear suddenly and require hospitalization due to the debilitating diarrhea and associated weight loss. The exact cause of Crohn's disease remains unknown, but most experts believe it to be an autoimmune disorder. It is also believed that there is a hereditary component. It has been reported that patients with Crohn's disease have a higher risk of developing colon cancer. Patients with Crohn's disease often undergo colonoscopy, flexible sigmoidoscopy, CT scans, MRI scans, capsule endoscopy, double-balloon endoscopy, and small bowel imaging to completely evaluate the digestive tract. Various anti-inflammatory drugs are used in an attempt to control the symptoms of Crohn's disease. These can include oral 5-aminosalicylates, corticosteroids, and immune system suppressing drugs such as Imuran, Purinethol, Remicade, Humira, Cimzia. These latter three drugs work by neutralizing an immune system protein known as tumor necrosis factor (TNF). Other drugs used to treat the symptoms of Crohn's disease include methotrexate, which is a cancer drug, and various cyclosporins including Gengraf, Neoral, Sandimmune, Astagraf XL, and hecora. Cyclosporins have the potential of serious side effects including kidney and liver damage, seizures and fatal infections. Tysabri and Entyvio are drugs that work by stopping certain immune cell molecules (integran) from binding to other cells in one's intestinal lining. The patients are also often treated with various antibiotics including Flagyl and ciprofloxacin. Various anti-diarrheal medications, pain relievers, iron supplements, vitamin B-12 shots, and the use of calcium and vitamin D supplements can sometimes help Crohn's disease. Surgery to remove damaged areas of the intestines can be temporarily helpful. Certain foods are recommended to be avoided such as dairy products and high fat containing foods. Other treatments include methods to reduce stress such as exercise, biofeedback, relaxation, and breathing exercises. Various herbal and nutritional supplements can be taken. Probiotics, fish oil, and prebiotics have not been found in studies to be helpful.

Further relevant information on Crohn's Disease may be found in the following publications, which are incorporated herein by reference in their entirety:
Abraham C, Cho J H (2009) Inflammatory bowel disease. N Engl J Medi 361:2066-2078.
Van Assche G, Dignass A, Panes J, Beaugerie L, Karagiannis J, Allez M, Ochsenkuhn T, Orchard T, Rogier G, Louis E, Kupcinskas L, Mantzaris G, Travis S, Stange E, European Crohn's and Colitis Organisation (ECCO) (2010) The second European evidence-based consensus on the diagnosis and management of Crohn's disease: definitions and diagnosis. J Crohns Colitis 4:7-27.
Baumgart D C, Sandborn W J (2007) Inflammatory bowel disease: clinical aspects and established and evolving therapies. Lancet 369:1651-1657.
Casellas F, Lopez-Vivancos J, Badia X, Vilaseca J, Malagelada J R (2001) Influence of inflammatory bowel disease on different dimensions of quality of life. Eur J Gastroenterol Hepatol 13:567-572.
Dalal, J (2013) Mesenchymal Stromal Cell (MSC) Therapy for Crohn's Disease. R. C. Zhao (ed.), Essentials of Mesenchymal Stem Cell Biology and Its Clinical Translation, DOI 10.1007/978-94-007-6716-4_15.
Clinical Trials.gov; U.S. National Institutes of Health. Evaluation of PROCHYMAL® Adult Human Stem Cells for Treatment-resistant Moderate-to-severe Crohn's Disease. ID # NTC 00482092 (May 19, 2015).
Clinical Trials.gov; U.S. National Institutes of Health. Evaluation of PROCHYMAL® for Treatment-refractory Moderate-to-severe Crohn's Disease. ID # NCT01233960 (May 19, 2015).
Jane Onken, M.D.; Dianne Gallup, MS; John Hanson, M.D.; Michael Pandak, M.D.; Linda Custer, PhD; Duke Clinical Research Institute, Durham, N.C. and Osiris Therapeutics, Baltimore, Md. Successful outpatient Treatment of Refractory Crohn's Disease using Adult Mesenchymal Stem Cells. (October 2006).
Loftus J R E V. Clinical epidemiology of inflammatory bowel disease: incidence, prevalence, and environmental influences. Gastroenterology. 2004, 126(6):1504-17.
Parashette K R, Makam R C, Cuffari C. Infliximab therapy in pediatric Crohn's disease: a review. Clin Exp Gastroenterol. 2010, 3:57-63.
Andres P G, Friedman L S. Epidemiology and the natural course of inflammatory bowel disease. Gastroenterol Clin North Am. 1999, 28(2):255-81. Vii.
Ciccocioppo R et al. Autologous bone marrow-derived mesenchymal stromal cells in the treatment of fistulising Crohn's disease. Gut. 2011, 60(6):788-798.
Garcia-Bosch O, Ricart E, Panes J. Review article: stem cell therapies for inflammatory bowel disease-efficacy and safety. Aliment Pharmacal Ther. 2010, 32(8):939-52.
Lazarus H M et al. Ex vivo expansion and subsequent infusion of human bone marrow-derived stromal progenitor cells (mesenchymal progenitor cells): implications for therapeutic use. Bone Marrow Transplant. 1995, 57(1):11-20.
Crohn's disease. National Institute of Diabetes and Digestive and Kidney Diseases. http://digestive.niddk.nih.gov/ddiseases/pubs/crohns/. Accessed Jun. 2, 2014.
Management of Crohn's disease in adults. Bethesda, Md.: American College of Gastroenterology. http://gi.org/guideline/management-of-crohn % e2%80%99s-disease-in-adults/. Accessed Jun. 2, 2014.
Peppercorn M A, et al. Clinical manifestations, diagnosis and prognosis of Crohn's disease in adults. http://www.uptodate.com/home. Accessed Jun. 2, 2014.
Farrell R J, et al. Overview of the medical management of mild to moderate Crohn disease in adults. http://www.uptodate.com/home. Accessed Jun. 2, 2014.
Farrell R J, et al. Overview of the medical management of severe or refractory Crohn disease in adults. http://www.uptodate.com/home. Accessed Jun. 2, 2014.
What is complementary and alternative medicine (CAM)? International Foundation for Functional Gastrointestinal Disorders. Http://www.iffgd.org/store/viewproduct/700. Accessed Jun. 25, 2014.
Kane S V, et al. Natalizumab for moderate to severe Crohn's disease in clinical practice: The Mayo Clinic Rochester experience. Inflammatory Bowel Diseases. 2012, 18:2203.
Ankrum J, Karp J M. Mesenchymal stem cell therapy: Two steps forward, one step back. Trends Mol Med. 2010 May, 16(5):203-209. Doi: 10.1016/j.molmed.2010.02.005
Duijvestein M, Hommes D W, Molendijk I (2013) Mesenchymal Stromal Cell Therapy in Crohn's Disease. Stem Cell Biology and Regenerative Medicine, DO110.1007/978-1-62703-200-1_11.
ISO Y, et al. Multipotent human stromal cells improve cardiac function after myocardial infarction in mice without long term engraftment. Biochem Biophys Res Commun 2007, 354:700-706 [Pubmed: 17257581].
Lee R H, et al. Intravenous hMSCs improve myocardial infarction in mice because cells embolized in lungs are activated to secrete anti-inflammatory protein TSG-6. Cell Stem Cell 2009, 5:54-63. [Pubmed: 19570514].
Ortiz L A, et al. Interleukin 1 receptor antagonist mediates the anti-inflammatory and antifibrotic effect of mesenchymal stem cells during lung injury. Proc Natl Acad Sci U.S.A. 2007, 104:11002-1107 [Pubmed:17569781].
Togel F, et al. Vasculotropic paracrine actions of infused mesenchymal stem cells are important to the recovery from acute kidney injury. Am J Physiol Renal Physiol. 2007, 292:F1626-F1635 [Pubmed: 17213465].

Treatment of Acute Orthopedic Soft Tissue Injuries.

In one embodiment of the invention, the therapeutic MSC products and associated methods of use disclosed herein provide a treatment for acute orthopedic soft tissue injuries, as described below in the Examples section. Soft tissue healing from acute injury requires a coordinated interplay among cells, growth factors, and extracellular matrix proteins. Central to this process is the MSC. This cell coordinates the repair response by recruiting other host cells in secreting growth factors and matrix proteins. MSCs are self-renewing multi-potent stem cells that can differentiate into various lineages of mesenchymal origin such as bone, cartilage, and tendon. MSCs regulate immune response and inflammation. These characteristics make the MSC have abilities to treat acute orthopedic soft tissue injuries. The beneficial effect of exogenous MSCs on wound healing has been documented on a variety of animal models and in clinical practice.

One relevant publication in regard to treatment of acute injury with MSCs is Caplan, A. I., et al., “The MSC: An Injury Drugstore,” Cell Stem Cell, 2011, Jul. 8; 9(1)11-15 [doi: 10.1016/j.stem.2011.06.008]. This publication by Caplan et al., and all publications referenced therein, are incorporated herein by reference in their entirety. Thus, Caplan et al. support a model that MSCs are clinically active at different tissue sites; that MSCs are pericytes and can be isolated from any vascularized tissue; and that MSCs secrete large quantities of a variety of bioactive molecules as part of their local trophic and immunomodulatory activities. Further, Caplan et al. propose that this specific MSC tissue “regulatory” phenotype arises as a consequence of broken or inflamed blood vessels at sites of tissue damage. This model does not exclude the possibility that pericytes naturally have an on/off cycle in the non-injured situation. Caplan et al. envision that this active phenotype can be adopted in addition to their “constitutive” phenotype in which, as perivascular cells, this population expresses MSC markers both in vivo and ex vivo and functionally exhibits multipotential ex vivo differentiation capabilities. According to this paradigm, in situations of vessel damage, the released pericytes become MSCs, are activated by the injury and respond to that tissue site by secreting a spectrum of bioactive molecules, (i.e., drugs) that serve to, first, inhibit any immune cell coming to survey the tissue damage and, thus, prevent autoimmune activities from developing. In addition, these secreted bioactive molecules, through their trophic activities, establish a regenerative microenvironment to support the regeneration and re-fabrication of the injured tissue. In this context, the MSCs serve as site-regulated, multidrug dispensaries, or “drugstores,” to promote and support the natural regeneration of focal injuries. If these injuries are large or occur in older individuals, the natural supply of MSCs must be supplemented by local or systemic delivery.

Other relevant publications in regard to wound repair and treatment of acute injury with MSCs are: Maxson, S., et al., “Concise Review: Role of Mesenchymal Stem Cells in Wound Repair,” Stem Cells Translational Medicine 2012, 1:142-149; and Uccelli, A., et al., “Mesenchymal Stem Cells in Health and Disease,” Nature Reviews Immunology 8:726-736 (1 Sep. 2008) [doi: 10.1038/nri2395]. These publications by Maxson et al. and Uccelli et al., and all publications cited therein, are incorporated herein by reference in their entirety.

Treatment of Liver Pathology Due to Alcohol Use.

In one embodiment of the invention, the therapeutic MSC products and associated methods of use disclosed herein provide a treatment for liver pathology due to alcohol use, as described below in the Examples section. It is known that most alcoholic liver damage is attributed to alcohol metabolism. Liver injury is believed to be caused by direct toxicity of metabolic byproducts of alcohol as well as inflammation induced by these byproducts, acetyl aldehyde and highly reactive molecules called free radicals. It has been reported that alcohol decreases antioxidants, especially glutathione; that alcohol decreases concentrations of vitamin A and E found in the liver; and that alcohol causes liver inflammation. This inflammation results in an imbalance of biological molecules including eicosanids, cytokines and endotoxins. Long-term alcohol consumption alters the balance of eicosanids in the liver by decreasing the production of cell protective prostaglandins and prostacyclins and increasing synthesis of harmful thromboxane B2 and Leukotriene B4. Alcohol increases liver cytokine production including tumor necrosis factor alpha (TNF-alpha). This cytokine is directly toxic to the liver. Alcohol causes an increase in circulating endotoxins from the intestines. Endotoxins directly cause liver inflammation. All of these alcohol-related biologic processes cause liver cells to break open or lyse resulting in a release of liver enzymes into the blood stream. These liver enzymes include alkaline phosphatase, aspartate aminotransferase, and alanine aminotransferase. These liver enzymes can be detected with a blood test, and increased values of these enzymes indicate liver damage. The rationale of utilizing mesenchymal stem cells to improve liver function is based on the following: improve the hepatic inflammatory microenvironment, inhibit the activation of liver cell death or apoptosis, replace damaged liver cells called hepatocytes, and promote the regeneration of residual hepatocytes. Extensive research supports the use of mesenchymal stem cells to promote liver health. Several human clinical trials have been published to evaluate the therapeutic potential of mesenchymal stem cells to treat liver pathology. Several published review articles detail the scientific data supporting the use of mesenchymal stem cells to help treat liver pathology, including the following publications: Berardis, S. et al., “Use of Mesenchymal Stem Cells to Treat Liver Fibrosis: Current Situation and Future Prospects,” World J. Gastroenterol 2015, Jan. 21, 21(3):742-758 [DOI: 10.3748/wjg.v21.i3.742]; Shiota, G., et al. (2016) “Progress in stem cell-based therapy for liver disease,” Hepatol Res, doi: 10.1111/hepr.12747; Vladislav, V., et al. “Concise Review: Therapeutic Potential of Mesenchymal Stem Cells for the Treatment of Acute Liver Failure and Cirrhosis,” Stem Cells, 2014, 32:2818-2823 [doi:10.1002/stem.1818]; Maher, J. J., “Exploring Alcohol's Effects on Liver Function,” Alcohol Health and Research World, 1997, 21(1):5-12. The foregoing publications by Berardis, et al., Shiota et al, Vladislav et al., and Maher, and all publications cited therein, are incorporated herein by reference in their entirety.

### EXAMPLES

The following examples further illustrate specific embodiments of the invention. However, the following examples should not be interpreted in any way to limit the invention. Thus, it is understood that modifications which do not substantially affect the activity of the various embodiments of this invention are also provided within the definition of the invention provided herein. Accordingly, the following examples are intended to illustrate but not limit the present invention.

### Example. Obtention of Autogenous MSCs

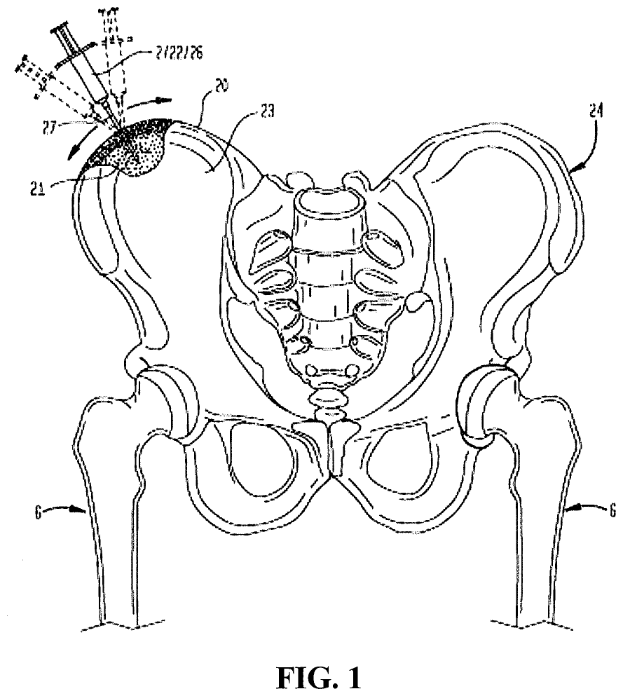

Referring to FIG. 1, autogenous MSCs (2) are multipotent stromal cells that can differentiate into a variety of cell types, including: osteoblasts (bone cells), chondrocytes (cartilage cells), myocytes (muscle cells) and adipocytes (fat cells). Autogenous MSCs (2), suitable for use in embodiments of the inventive method, are typically obtained from bone marrow (21) or fractions thereof; however, this is not intended to limit obtention of autogenous MSCs (2) suitable for use with the inventive method solely from bone marrow (21) and it is understood that autogenous MSCs (2) may be isolated from other tissues such as: peripheral blood, synovium, periosteum, skeletal muscle, or adipose tissue. Additionally, as to certain embodiments, it is understood that autogenous MSCs (2), whether from bone marrow (21) (or fractions thereof) or other tissue sources, can optionally be expanded in media to obtain an amount of autogenous MSCs (2) for use in embodiments of the inventive method.

Typically, bone marrow aspirates can have a cellularity of marrow (21) of about 15 to about 30 million mononuclear cells per milliliter (“MNC/mL”), whereas less than 10 million MNC/mL usually means a rather diluted sample provided the donor was healthy and the marrow not fibrotic. The average colony forming unit-fibroblasts (“CFU-f”) content of healthy human bone marrow is about 100 CFU-f/million MNC. Accordingly, bone marrow concentrate (22) (“BMC”) resulting from aspirating bone marrow (21) can contain a few thousands (about 1500 to about 3000) CFU-f/mL of bone marrow (21).

As to particular embodiments, autogenous MSCs (2) obtained from bone marrow (21) or adipose tissue may be utilized in embodiments of the inventive method as autogenous BMC (22) or adipose-derived stromal vascular fraction (SVF) without further processing; although autogenous MSCs (2) can be contained or entrained in other biocompatible materials or compositions known in the relevant art. One preferred source of autogenous BMC (22) can be the bone marrow (21) of the ilium (23) of the greater pelvis (24); although autogenous BMC (22) obtained from other sites may be utilized. Adipose tissue is typically suctioned from the abdomen but other anatomical locations can also be used. Adipose-derived SVF, as has been described in the art, may be used; for example, as described by Oberbauer, E., et al., “Enzymatic and Non-Enzymatic Isolation Systems for Adipose Tissue-Derived Cells: Current State of the Art,” Cell Regeneration, 215, 4:7, the disclosure of which is incorporated herein by reference in its entirety.

### Example. Bone Marrow Collection and Processing

Following is an illustrative example of bone marrow collection and processing to BMC, which was carried out as described in Pettine, K. A., U.S. Pat. No. 9,408,874 (Aug. 9, 2016), which is incorporated herein by reference in its entirety. Accordingly, 60 cc of bone marrow aspirate was collected over ACD-A as needed per process. The marrow was processed using the bone marrow concentration system according to the detailed protocol. The patient was given IV antibiotics and placed prone on an image table. Intravenous Versed and Fentanyl was administered and the skin was anesthetized with buffered 1% Lidocaine. The aspirator was rinsed and the syringes were transferred with heparin solution, approximately 1000 U/ml. The heparin solution coated the inner surface of the 60-cc aspiration needle and trephine needle. The remaining heparin was expelled from the syringe. 6 cc of ACD-A was aspirated into the 60-cc syringe. Bone marrow was aspirated from the posterior iliac crest when the patient was positioned prone on a fluoro table. The right iliac wing was prepped and draped according to standard surgical protocols. A trephine needle and 60 cc syringe was used to remove the marrow. The surgeon inserted the trephine needle percutaneously through the skin until the bony surface of the iliac crest was felt. Using a mallet, the needle was then inserted to a depth of 3-4 cm into the crest. This was accomplished with fluoroscopic guidance. A 60-cc syringe containing 6 ml of acid citrate dextrose anticoagulant solution (ACD-A) (10% of the final volume) was attached to the needle. The marrow was aspirated by pulling the plunger back and allowing the syringe to fill to the 10-cc level. The needle was repositioned by advancing 1.5-2 cm and an additional 10 cc of aspirate was obtained. This process was repeated until 60 cc of iliac aspirate was obtained. Once the final marrow volume was reached, the solution was mixed by gentle rocking of the syringe as the syringe was rotated on its long axis. The marrow was then ready for processing. The marrow was mixed with anticoagulant solution by gently turning the syringe after each 10 cc of aspirate collection. The extracted marrow was placed in an isolating canister and loaded into the centrifuge. The marrow was centrifuged for about 12 minutes at about 3200 rpm. The processed marrow was drawn with a syringe from the centrifuge and then rocked while rotating the syringe on its long axis. The syringe was then presented to a sterile field. The amount of bone marrow concentrate removed from the centrifuge equaled the amount to be used. The cell delivery media was pre-mixed and aliquoted to 1 cc, composing of 0.5 cc of ACD-A and 0.5 cc of dextrose (50%). The delivery media was injected into a closed vial containing the cell components slowly, in order to homogenize the mixture and incorporate oxygen and turbidity mixing in a closed, sterile system. Optionally, one or more fillers of the types used in the art may be added, but are not required. The resulting mixture was then used for treatment of skin tissue without delay. Typically, the entire procedure from beginning of BMA collection to treatment of a patient's skin tissue is performed in less than one hour.

Following is a second illustrative example of bone marrow collection and processing to BMC. Bone marrow aspirate (BMA, 55 mL) was collected over acid citrate dextrose-anticoagulant (ACD-A, 5 mL) from the patient's posterior iliac crest. The procedure was performed with IV sedation consisting of Versed and Fentanyl. Positioning of the trephine needle in the iliac wing was confirmed by fluoroscopy. BMA was collected in a 60 mL syringe in a series of discrete pulls on the plunger (targeting a collection of 5-10 mL per pull), with repositioning of the needle tip between pulls based on the reported enrichment of progenitor cells. The BMA was processed using a centrifuge to produce a bone marrow concentrated cell preparation. Typically, a BMC volume of 7 mL (6 mL for injection and 1 mL for cell analysis) was drawn from the processing device.

Cell analysis and characterization of 20 out of the 26 patients' BMC samples were performed. An aliquot (1 mL) of each subject's BMC was packed in a shipping container with 5° C. cold packs and shipped overnight to the cell analysis laboratory. The samples were received and processed immediately to determine total nucleated cell count and viability. The BMC was diluted in phosphate buffered saline with 2% fetal bovine serum and subjected to a gradient separation in order to deplete red blood cells. Analysis of the recovered cells included performing colony forming unit-fibroblast and osteogenic (CFU-F and CFU-O, respectively) assays and phenotypic analysis by flow cytometry. For phenotype analysis, fresh (non-cultured) BMC cells were stained with a series of rabbit anti-human monoclonal antibodies for a hematopoietic lineage-committed (non-progenitor) panel of markers including CD2, 3, 8, and 11b (APC-Cy7), CD34 (PE), CD90 (FITC), and CD105 (APC), as well as appropriate isotype controls. Isotype, single color stain, and four-color stain samples were analyzed. The CFU-F assay was performed by creating a dilution series (in culture medium with 5% FBS and 1% antibiotics) of each cell preparation at concentrations of 50,000 to 500,000 total nucleated cells (TNC) per well in standard 12-well plates. The plates were placed in an incubator at 37° C., 5% CO₂ and 100% humidity for 72 hours when the medium was replaced. Medium was replaced every 3 days. After 9 days in culture, wells were gently washed with PBS, fixing the colonies/cells with methanol, staining the attached cells with Crystal Violet, rinsing with water and air-drying the plates. Visualization and counting of the colonies was done with an inverted microscope. Colonies containing 20 or more cells were scored as a CFU-F. The CFU-O assay was performed identically as CFU-F, but after 9 days the medium was changed to an osteogenic induction medium for an additional 9 days with complete medium change every 3 days. On day 18, the wells were washed with PBS, then fixed for 15 minutes in 2% formalin solution and co-stained for alkaline phosphatase activity and calcified extracellular matrix.

Univariable data comparisons (pain scores by time, patient age, number of levels injected, or CFU-F concentration; CFU-F frequency by patient age or CFU-O) were analyzed by two-tailed Student's t-test with a 95% confidence interval (=0.05). Multivariable data were evaluated by analysis of variance (ANOVA) using JMP 9 statistical analysis software (SAS Institute, Cary, N.C.).

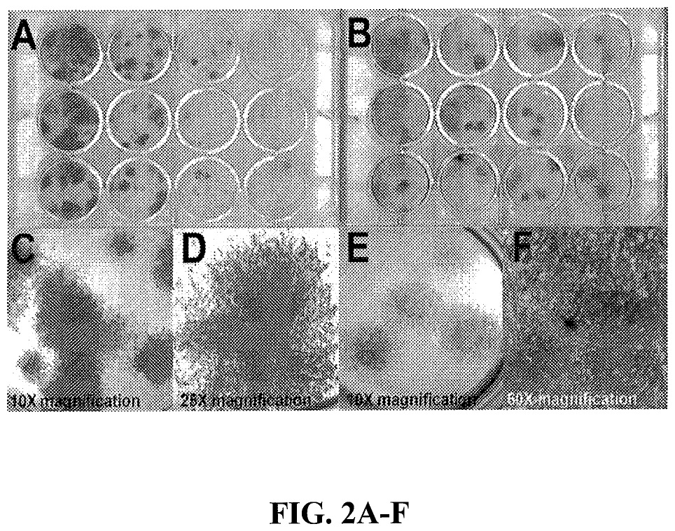

Fresh BMC aliquots were analyzed within 24 hours of the procedure. The average TNC concentration, cell viability, CFU-F frequency, CFU-O frequency, and CD marker phenotypic analyses are reported in Table 1. TNC and CFU-F per mL of BMC injectate yields were consistent with published manufacturer's data. The average CFU-O frequency and concentration were slightly higher, but within statistical error compared to CFU-F. All BMC samples yielded robust CFU-F formation after 9 days in culture with a virtually identical yield and frequency of CFU-O. CFU-F and CFU-O stained colonies are illustrated in FIG. 1. Alkaline phosphatase activity is displayed in blue, while mineralization resulted in red coloration of colonies. The statistical correlation between CFU-F and CFU-O is shown in FIG. 2A and demonstrates that 18 of the 20 CFU-O samples analyzed fall within the 95% confidence interval of CFU-F. This indicates that not only do the samples possess a classical characteristic of MSCs (CFU-F in primary in vitro culture), but they also have the capacity to differentiate at nearly a 1-to-1 correlation with CFU-F.

TABLE 1
Average Cell Viability, Total Nucleated Cells (TNC), Total and Frequency
of CFU-F/CFU-O and CD Marker Phenotypes in fresh Bone Marrow Concentrate.

| Cell Viability at 24 hours | 98.1 (±1.2) % | TNC/mL in BMC 121 (±11) × 10⁶ | |
|---|---|---|---|
| Cell Phenotype Subpopulation | % of TNC | Subpopulation Concentration in BMC | |
| Frequency | | (cells/mL) | |
| CFU-F | 0.0025% | 3,019 (±491) | per mL |
| CFU-O | 0.0027% | 3,225 (±418) | per mL |
| Lineage⁻ Cells (CD 2⁻/3⁻/8⁻/11b⁻) | 25.89% | 31.5 × 10⁶ | per mL |
| Lineage⁻/CD34⁺ | 1.397% | 1.69 × 10⁶ | per mL |
| Lineage⁻/CD34^{High}/CD90+/CD105+ | 0.0007% | 802 | per mL |
| Lineage⁻/CD34^{Low}/CD90+/CD105+ | 0.0040% | 4,832 | per mL |
| Lineage⁻/CD34⁻/CD90+/CD105+ | 0.0049% | 5,914 | per mL |

A substantial fraction of Lineage⁻ (cells not committed or differentiated toward a hematopoietic lineage) cells were positive for CD90, CD105 and CD34, which are common markers for mesenchymal and hematopoietic stem cells. CD34 expression was observed as three distinct populations: CD34^{High} CD34^{Low}, and CD34⁻. As MSCs have been reported universally to express both CD90 and CD105, the percentages of cells from each of the three CD34 subpopulations that were also Lineage−/CD90⁺/CD105⁺ were compared to the CFU-F frequency for each individual BMC sample in an attempt to define a phenotypic population of interest. As listed in Table 2, the average CFU-F frequency was 0.0025%, or approximately 25 per million TNC. The Lineage⁻/CD34^{High}/CD90⁺/CD105⁺ population represented only 0.0007% of nucleated cells, while the Lineage⁻/CD34^{Low}/CD90⁺/CD105⁺ (0.0040%) and Lineage⁻/CD34/CD90⁺/CD105⁺ (0.0049%) populations exceeded the CFU-F frequency and could encompass the MSC population. A linear regression was performed on CFU-F frequency versus Lineage⁻/CD90⁺/CD105⁺ phenotypes by CD34 expression (FIG. 2B). Although none of the populations fit to the CFU-F unity line within statistical error (R²>0.9), the linear fit of Lineage⁻/CD34^{Low}/CD90⁺/CD105⁺ most closely matched CFU-F. This method of analysis did not eliminate Lineage⁻/CD34⁻/CD90⁺/CD105⁺ as a candidate population of CFU-F, as it was within one standard deviation of the CFU-F line for the majority of frequencies.

TABLE 2
Average Pre- and Post-treatment Pain (Oswestry Disability Index, ODI)
and QOL (Visual Analogue Scale, VAS, 0-100) Scores. Statistically
significant differences from Pre-treatment score: p ≤ 0.0001
(*), p < 0.005 (**), p < 0.01 (***). Statistically significant
differences between compared populations: p < 0.005 (#), p < 0.01 (##).

| Patient | | Pre- | | | |
|---|---|---|---|---|---|
| Population | Assessment | treatment | 3 month | 6 month | 12 month |
| All Subjects | ODI | 56.5 | 22.8* | 24.4* | 25.0* |
| (n = 26) | VAS | 79.3 | 29.2* | 26.3* | 33.2* |
| One-Level | ODI | 56.5 | 18.4* | 19.8* | 26.2** |
| Injection | VAS | 78.5 | 23.8* | 20.2* | 31.4* |
| (n = 13) | | | | | |
| Two-Level | ODI | 55.5 | 27.4** | 29.3** | 22.7* |
| Injections | VAS | 79.4 | 34.8* | 32.7* | 33.0* |
| (n = 13) | | | | | |
| Age ≤40 | ODI | 57.1 | 18.2* | 20.6* | 25.1** |
| (n = 14) | VAS | 83.4 | 24.6* | 23.5* | 32.3* |
| Age >40 | ODI | 55.8 | 27.8** | 28.5** | 24.8** |
| (n = 12) | VAS | 74.8 | 34.2** | 29.2** | 34.5 |
| CFU-F per | ODI | 54.2 | 33.7*** | 36.3 | 26.3** |
| mL <2000 | VAS | 80.4 | 46.4** | 36.7** | 34.5** |
| (n = 9) | | | | | |
| CFU-F per | ODI | 59.3 | 14.8*^{,#} | 13.5*^{,#} | 17.6* |
| mL >2000 | VAS | 82.0 | 17.5*^{,#} | 10.8*^{,#} | 25.5* |
| (n = 11) | | | | | |

### Example. Expanding Human MSCs (hMSCs)

An embodiment of the disclosed invention provides MSC products, and associated methods, for treating autoimmune conditions and for promoting rapid healing from acute traumatic soft tissue orthopedic injuries, and the like, using autogenous or allogeneic MSCs, wherein the MSCs have been cultured and/or culture-expanded. Evidence exists that this may enhance the efficacy as well as safety of hMSC therapeutics. Various techniques and methods for expanding the MSCs are well known and have proliferated in the art, including all aspects of preparing the culture media, cell bioprocessing protocols, and related steps, while maintaining the therapeutic and differentiation capacity of the MSCs. For the purpose of this invention, the following publications encompass the techniques and methods for expanding the MSCs prior to their use in the MSC products and associated methods of the invention. Thus, the disclosures of the following publications are hereby incorporated by reference in their entirety:
Jung, S., et al., “Ex Vivo Expansion of Human Mesenchymal Stem Cells in Defined Serum-Free Media,” Stem Cells International, Volume 2012, Article ID 123030, pages 1-21.
Bruedigam, C., et al., “Basic Techniques in Human Mesenchymal Stem Cell Cultures: Differentiation into Osteogenic and Adipogenic Lineages, Genetic Perturbations, and Phenotypic Analyses,” Curr. Protoc. Stem Cell Biol. 17:1H.3.1-1H.3.20.
Battula, V. L., et al., “Human placenta and bone marrow derived MSC cultured in serum-free, b-FGF-containing medium express cell surface frizzled-9 and SSEA-4 and give rise to multilineage differentiation,” Differentiation (2007) 75:279-291.
Ikebe, C., et al., “Mesenchymal Stem Cells for Regenerative Therapy: Optimization of Cell Preparation Protocols” BioMed Research International, Volume 2014, Article ID 951512, pages 1-11.

### Example. Obtaining Allogeneic-Derived MSCs

An embodiment of the disclosed invention provides MSC products, and associated methods, for treating autoimmune conditions and for promoting rapid healing from acute traumatic soft tissue orthopedic injuries, and the like, using autogenous or allogeneic MSCs. Regarding allogeneic MSCs, methods and sources of obtention have proliferated in the art in recent years. Illustratively, the allogeneic MSCs may be obtained from the iliac wing, the removed bone from a total hip or knee replacement, or from other appropriate aspiration sites from one or more screened donors. For the purpose of this invention, the foregoing publications above by Jung et al., Bruedigam et al., Battula et al., and Ikebe et al. encompass the techniques and methods for obtaining the allogeneic MSCs of the invention; and, accordingly, the disclosures of these four publications are hereby incorporated by reference in their entirety for that purpose. Additionally, various companies have emerged that provide specialized culture media to produce MSCs that have the ability to maximize fibroblast differentiation and increase collagen fiber production, elastin fiber production, and glycosaminoglycan production, as well as to maximize production of various growth factors, including bFGF, KGF-2, IGF-1, EGF, and SOD-1. For the purpose of this invention, the following is an illustrative list of companies that provide specialized culture media to produce the allogeneic MSCs of the invention: PromoCell, Life Cell Technology, StemPro®, StemMACS™, and Cell Applications Inc.

### Example. Use of Jamshidi Bone Biopsy Needle to Aspirate Bone Marrow

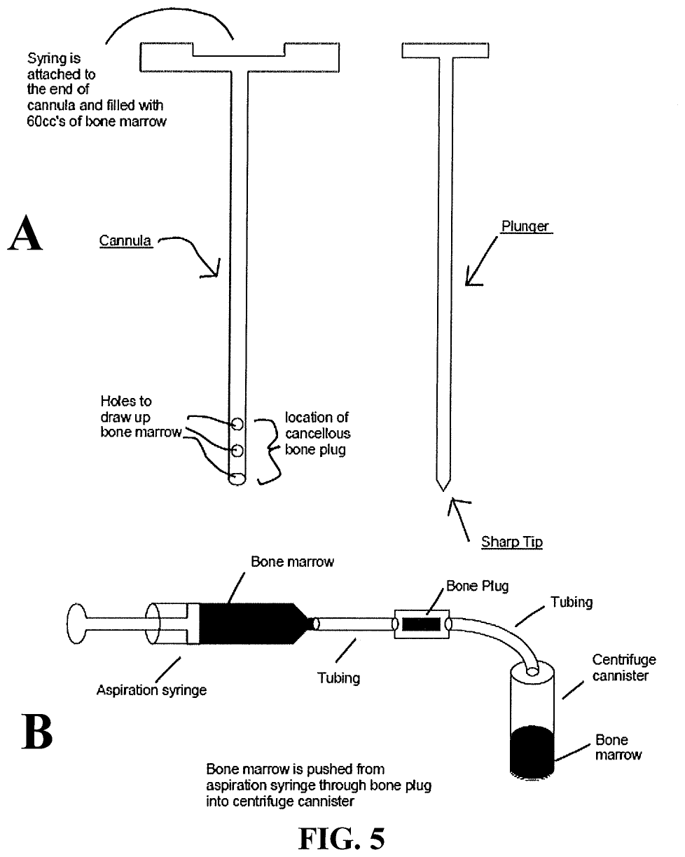

Reference is made to FIG. 5. Plunger fits into cannula to begin penetration into the bone. The sharp tip of plunger penetrates cortical bone. Needle is inserted 1 to 2 cm into cancellous bone then plunger is withdrawn. Cannula is inserted additional 1-2 cm to fill tip of cannula with cancellous bone plug. Bone marrow is then aspirated into a syringe attached to the cannula with discrete pulls on the syringe. Cannula is moved 1-2 cc after each 10 cc of bone marrow is aspirated. The bone marrow is aspirated through the bone plug. The bone plug is replaced three times during procedure.

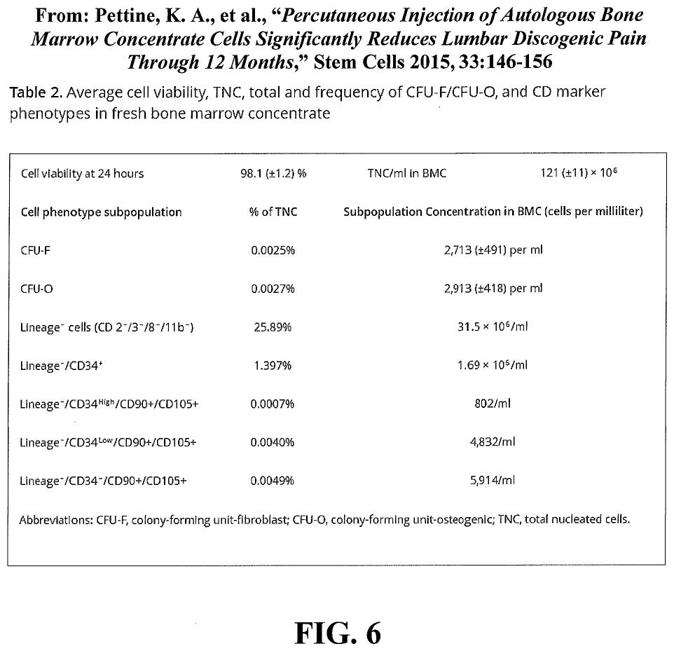

In one embodiment of the invention, it has been surprisingly and unexpectedly discovered that pulling the bone marrow through one or more cancellous bone plugs increases the number of mesenchymal stem cells being aspirated into the syringe. To maximize cell counts the bone marrow is pushed through the bone plugs a second time when transferring the bone marrow from the aspiration syringe into the centrifuge canister. Laboratory tests show this novel technique more than doubles the mesenchymal stem cells found in the bone marrow concentrate after centrifuging the bone marrow, relative to the traditional method of aspirating bone marrow. FIG. 6 is a copy of published tabular data (from Pettine, K. A., et al., “Percutaneous Injection of Autologous Bone Marrow Concentrate Cells Significantly Reduces Lumbar Discogenic Pain Through 12 Months,” Stem Cells 2015, 33:146-156; incorporated herein by reference in its entirety), which shows what the cell counts typically are with the traditional method of bone marrow aspiration.

### Example. Further Example of Collection of BMC, Filtering Through Cancellous Bone, and Bathing in a Glucose-Containing Sauce

Bone marrow is aspirated into a 100-cc syringe with 8 cc of anticoagulant, to obtain 60 cc of bone marrow aspirate, as described earlier. A 2 mm by 8-10 mm cancellous iliac wing bone plug is placed into a three-way stopcock. The 60 cc of bone marrow aspirate is pushed with pressure through a filter and then through the stopcock containing the cancellous bone plug into a 100-cc specialized container designed to be placed into a centrifuge. The bone plug can be replaced several times during the transfer of bone marrow aspirate to the specialized centrifuge container. Laboratory analysis indicates this technique greatly increases the mesenchymal stem cell counts. After centrifuging the bone marrow concentrate, the specialized container holds the 60 cc of aspirate that is now in layers. The plasma layer containing platelets is in the top layer. The bottom layer holds the red blood cells. In between is a layer containing the nucleated cells which also includes the mesenchymal stem cells. This layer has approximately 10 cc of volume. This material is placed into a 20-cc syringe. To this 10 cc of nucleated cells is added about 0.5 cc and up to about 1 cc of 50% glucose by volume. The glucose containing bone marrow concentrate is gently stirred for from about one up to about ten minutes to allow maximal cellular uptake of the glucose. The purpose is to maximize the energy available for the mitochondria to maximally produce ATP. The bone marrow is then pushed gently through an 18 Micron filter into a second syringe which is then placed into the patient's IV.

### Example. Treatment of Various Diseases and Conditions with Intravenous Infusion of Bone Marrow Concentrate (BMC)

In an embodiment of the invention, patients suffering from one or more of the following conditions were successfully treated intravenously with the MSC-containing BMC of the invention: Fibromyalgia, Multiple Sclerosis, Parkinson's, Rheumatoid Arthritis, Crohn's Disease, and elevated liver enzymes from alcohol use. All of the patients reported favorable results with no adverse events. Further details are given in the following examples.

### Example. Treatment of Fibromyalgia

Fibromyalgia can result in a chronic debilitating condition impacting all aspects of a patient's life. This condition has no current cure. All of the treatments utilized for fibromyalgia are simply to control the symptoms and not cure the underlying problem. Most experts agree this is a rheumatologic condition associated with numerous autoimmune disorders.

In an embodiment of the invention, patients suffering from fibromyalgia were successfully treated intravenously with the MSC-containing BMC of the invention. Thus, a small number of patients with multi-year histories of debilitating fibromyalgia have been treated with the use of intravenous MSC-containing BMC. This involves placing the BMC through a filter (Hemo-Nate® syringe infusion set) which removes the cell aggregates and particulates (18 μM or larger) prior to the slow infusion of the BMC. No adverse effects from this therapy are observed.

Thus, the procedure involves placing the MSCs into the peripheral vein of a patient as described in the following illustrative example. To start the procedure, an IV is started, typically in an arm of the patient. Antibiotics are placed into the IV and a small dose of IV Versed is given for relaxation. The patient is transported a short distance to the procedure room to lay on a table similar to a massage table. The patient is placed on their abdomen and additional medication is given consisting of more IV Versed and fentanyl (narcotic). At this point, most people are asleep or extremely relaxed. The Versed also gives the patient amnesia for a brief time that the medication is in their blood stream. Once the patient is completely relaxed or asleep, the skin area around the posterior iliac wing is sterilized with betadine and draped sterilely. The skin is anesthetized with buffered 1% xylocaine. Utilizing fluoroscopic control, a Jamshidi needle is placed into the patient's posterior iliac wing. At this point, anywhere from 60 to 120 mL of bone marrow is carefully extracted. This entire procedure is performed with the patient asleep or highly relaxed and having amnesia. At this point, the patient is placed in a wheelchair and transported back to a comfortable lounge chair to sleep or be highly relaxed for approximately one half hour. During this time the bone marrow aspirate is placed in a centrifuge for approximately 15 minutes. This separates the bone marrow aspirate into various cell layers with red blood cells being at the bottom of the tube and plasma at the top. A small layer in the middle contains all of the nucleated cells including the MSCs. This layer is extracted and placed through a Hemo-Nate® Syringe Infusion Set. This filter removes all of the aggregates and particulates that are 18 μM (Micrometers) or larger. The mesenchymal stem cell layer is placed into the patient's IV at a rate of approximately 1 mL per minute. The MSCs then travel to the capillary bed of the patient's lungs where they remain for a few hours and then travel throughout the body. The MSCs are attracted to areas of inflammation in the body. They also release various growth factors and communicate to other cells to modulate the immune system. The IV is removed and the patient is transported home with a driver. Usually, the patient is sore from the removal of bone marrow for approximately 24 hours and may feel relaxed or sleepy for several hours following the procedure.

A 55 year old female patient was diagnosed with fibromyalgia in October of 2004. As a United States Air Force Master Sergeant, she began suffering from chronic pain and fatigue in 1998. These symptoms progressed into headaches, joint swelling and immobility, sensitivity to light and sound, mood swings, and deep depression. To combat these debilitations, the patient took various medications such as Vicodin, hydromorphone (Dilaudid), Tramadol, cyclobenzaprine (Flexeril), duloxetine (Cymbalta), ketoconazole cream (2%), and clonazepam. After finding that she was not getting the necessary relief, the patient pursued an alternative treatment, and was treated by the inventor in July of 2014 in accordance with the foregoing procedure via IV MSC therapy. Three weeks after the treatment the patient claims she was able to notice a difference in her pain and day-to-day function. Five months after her IV treatment, she was completely off of her previous medications. Prior to her treatment, she was mostly bed-ridden and unable to perform most daily activities. Now, one year after her first IV injection, the patient's health has improved tremendously, and she is able to live a more active and healthy lifestyle.

### Example. Treatment of Multiple Sclerosis (MS)

MS can result in a chronic debilitating condition, impacting all aspects of the patient's life. This condition has no current cure. All of the current treatments utilized for MS are simply to control the symptoms and not cure the underlying problem. Most experts agree that this condition is an autoimmune disorder. In an embodiment of the invention, patients suffering from MS were successfully treated intravenously with the MSC-containing BMC of the invention. Thus, a small number of patients suffering from years of debilitating symptoms of MS were treated via the intravenous infusion of the MSC-containing BMC of the invention, resulting in significant improvement in the patients' health, with no adverse effects. The procedure for obtaining the BMC and placing it via IV infusion into the patients' peripheral veins is similar to the procedure described in the above example for treatment of fibromyalgia.

### Example. Treatment of Parkinson's Disease

Parkinson's Disease can result in a chronic debilitating condition, impacting all aspects of the patient's life. This condition has no current cure. All of the treatments utilized for Parkinson's Disease are simply to control the symptoms and not cure the underlying problem. Most experts agree that this condition is an autoimmune disorder. In an embodiment of the invention, patients suffering from Parkinson's Disease were successfully treated intravenously with the MSC-containing BMC of the invention. Thus, a small number of patients suffering from years of debilitating symptoms of Parkinson's Disease were treated via the intravenous infusion of the MSC-containing BMC of the invention, resulting in significant improvement in the patients' health, with no adverse effects. The procedure for obtaining the BMC and placing it by IV infusion into the patients' peripheral veins is similar to the procedure described in the above example for treatment of fibromyalgia.

### Example. Treatment of Crohn's Disease

In an embodiment of the invention, patients suffering from Crohn's Disease were successfully treated intravenously with the MSC-containing BMC of the invention. Thus, a small number of patients suffering from years of debilitating symptoms of Crohn's Disease were treated via the intravenous infusion of the MSC-containing BMC of the invention, resulting in significant improvement in the patients' health, with no adverse effects. The procedure for obtaining the BMC and placing it by IV infusion into the patients' peripheral veins is similar to the procedure described in the above example for treatment of fibromyalgia.

A 56-year old male patient was diagnosed with Crohn's disease in 2006. Since their diagnosis, the patient has suffered from symptoms such as nausea, cyclic vomiting, blood in stool, cramping, general abdominal pain, and headaches. To alleviate these symptoms, the patient at one point was taking mercaptopurine (30 mg daily), methadone (40 mg daily), and indicated post-treatment that they had been receiving infliximab therapy every 16 weeks. Prior to any stem cell treatment, the patient was driven or taken by ambulance to the emergency room every 2-3 months to attend the more intense symptoms. In December of 2014, the patient underwent intravenous mesenchymal stem cell therapy, carried out by the inventor, in order to find alternative relief. Six months after receiving the IV treatment, the patient indicated that they had found tremendous alleviation of their symptoms and had not been to the emergency room since. While they still sought infliximab therapy every 16 weeks following the stem cell procedure, the patient had reduced their methadone dosage to 10 mg daily from 40 mg daily and ceased taking mercaptopurine. After completing a general health survey six months post-treatment, the patient's scores indicate that their physical, emotional, and social health had all improved, as well as their pain threshold, day-to-day functioning, and other daily activities. The data acquired from this patient reveals that they have achieved some relief from their Crohn's disease symptoms through this treatment after six months and have had no complications since.

### Example. Treatment of Acute Orthopedic Soft Tissue Injuries

In an embodiment of the invention, patients suffering from acute orthopedic soft tissue injuries were successfully treated intravenously with the MSC-containing BMC of the invention, and experienced an energy boost. Following is one illustrative example. Men who participate in the National Football League (NFL) represent an elite group of highly trained athletes. They are subjected to daily and intense force vectors through a 21-week season, not including the preseason. Acute orthopedic soft tissue injuries occur to 100% of NFL athletes throughout their season. The inventor has performed an IV infusion of 20 mL of the BMC of the invention on five occasions to NFL defensive backs and linebackers. Three of these took place 11 days before Super Bowl 50. On all occasions the players reported acute orthopedic soft tissue injuries to the ankle, knee, or shoulder prior to the IV infusion of BMC. Within days following the IV infusion of 20 mL of BMC, all of the NFL players reported dramatic improvement in their injuries and an overall subjective energy boost. In addition, over 300 patients have been treated with an IV infusion of BMC in a volume ranging from 10 mL to 20 mL of BMC at one treatment. These patients typically report an overall energy boost and marked improvement in pain and function within days following the procedure lasting months. The procedure for obtaining the BMC and placing it by IV infusion into the patients' peripheral veins is similar to the procedure described in the above example for treatment of fibromyalgia.

### Example. Treatment of Liver Pathology Due to Alcohol Use

In an embodiment of the invention, patients suffering from liver pathology due to alcohol use were successfully treated intravenously with the MSC-containing BMC of the invention, exhibiting marked improvement in liver function tests. Following is one illustrative example. A 61-year-old businessman with a 40-year history of moderate alcohol consumption was seen by the inventor, Dr. Pettine. He gave a history of consuming 2 to 4 drinks per night and more on the weekends. He recently had undergone his annual physical examination with liver function tests performed on Dec. 14, 2014. He underwent an IV infusion of the BMC of the invention by Dr. Pettine on Jan. 5, 2015 and had his liver function test repeated on Mar. 15, 2015. He reported no change in his alcohol consumption from Dec. 14, 2014 through Mar. 15, 2015. The only change in his history was the IV infusion of 20 mL of BMC on Jan. 5, 2015. His liver function tests improved from being abnormal on Dec. 14, 2014: alkaline phosphatase was 122 with a normal range of 34 to 104, aspartate aminotransferase levels were 50 with a normal range between 13 to 39, alanine aminotransferase was 67 with a normal range being 7 to 52. His lab values on Mar. 15, 2015 were all within normal values with an alkaline phosphatase of 64.2, an aspartate aminotransferase of 35.2, and an alanine aminotransferase of 37.9. The procedure for obtaining the BMC and placing it by IV infusion into the patient's peripheral veins is similar to the procedure described in the above example for treatment of fibromyalgia.

As can be easily understood from the foregoing, the basic concepts of the present invention may be embodied in a variety of ways. The invention involves numerous and varied embodiments of treatments of the conditions described herein, and the like.

As such, the particular embodiments or elements of the invention disclosed by the description or shown in the figures or tables accompanying this application are intended to be exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “treatment” should be understood to encompass disclosure of the act of a “treating”, whether explicitly discussed or not, and, conversely, were there effectively disclosure of the act of “treating”, such a disclosure should be understood to encompass disclosure of a “treatment” and even a “means for treating.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition each definition hereby incorporated by reference.

All numeric values herein are assumed to be modified by the term “about”, whether or not explicitly indicated. For the purposes of the present invention ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes, for example, the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

It is to be understood that, as used herein, the grammatical conjunction “and/or” refers throughout to either or both of the stated possibilities.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Moreover, for the purposes of the present invention, the term “a” or “an” entity refers to one or more of that entity unless otherwise limited. As such, the terms “a” or “an”, “one or more” and “at least one” can be used interchangeably herein.

Also, for the purposes of the present invention, it is to be understood that the volume units “mL” and “cc” are considered to be approximately equal. As such, these units can be used interchangeably herein.

Thus, the applicant(s) should be understood to claim at least: i) a treatment of autoimmune conditions and acute traumatic soft tissue resulting from orthopedic injuries, and the like, with autogenous and/or allogeneic mesenchymal stem cells herein disclosed and described, ii) the related methods disclosed and described, iii) similar equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

While the disclosure has been illustrated and described in detail in the figures and foregoing description, the same is to be considered as illustrative and not restrictive in character, it being understood that only selected embodiments have been shown and described and that all changes, modifications and equivalents that come within the spirit of the disclosures described heretofore and/or defined by the following claims are desired to be protected. It will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. In addition, all publications cited herein are indicative of the level of skill in the art and are hereby incorporated by reference in their entirety as if each had been individually incorporated by reference and fully set forth.

What is claimed is:

1. A therapeutic method for treating a patient suffering from one or more conditions, said method comprising the steps of:
(a) obtaining mesenchymal stem cells;
(b) mixing the mesenchymal stem cells with a premixture comprising a predetermined quantity of an aqueous anticoagulant solution, a predetermined quantity of a dextrose solution, and a predetermined quantity of phosphate buffered saline, to form a first mixture;
(c) bathing said first mixture for a period of time in a glucose-containing sauce to form a second mixture; and,
(d) administering a therapeutically effective amount of the second mixture to the patient;
wherein the one or more conditions the patient is suffering from are one or more of an autoimmune disease, acute traumatic soft tissue orthopedic injuries, or elevated liver enzymes from alcohol.

2. The method of claim 1, further comprising obtaining bone marrow concentrate containing said mesenchymal stem cells.

3. The method of claim 2, further comprising aspirating bone marrow to obtain said bone marrow concentrate.

4. The method of claim 3, wherein aspirating the bone marrow further includes passing the bone marrow one or more times through one or more plugs of cancellous bone.

5. The method of claim 3, wherein the bone marrow concentrate is obtained from the bone marrow by centrifugation.

6. The method of claim 5, further comprising the step of filtering the bone marrow concentrate through a microfilter of a size that is effective in removing bone debris, aggregates, particulates, and/or non-cellular contaminants.

7. The method of claim 6, wherein the size of the microfilter is 18 μm.

8. The method of claim 3, further comprising aspirating bone marrow of an ilium of a pelvis.

9. The method of claim 1 wherein the autoimmune disease is one or more than one of Parkinson's Disease, Multiple Sclerosis, Fibromyalgia, Rheumatoid Arthritis, and Crohn's Disease.

10. The method of claim 1, wherein the glucose-containing sauce comprises a glucose solution having a glucose concentration of from about 25% to about 75% by volume.

11. The method of claim 10, wherein the glucose solution includes glucose dissolved or suspended or emulsified in an aqueous medium, or in a mixture of an aqueous medium and one or more other art-recognized suitable media.

12. The method of claim 1, wherein the bathing period of time is between about 1 minute and about 5 minutes, between about 5 minutes and about 10 minutes, between about 10 minutes and about 15 minutes, between about 15 minutes and about 20 minutes, between about 20 minutes and about 25 minutes, or between about 25 minutes and about 30 minutes, or is longer than about 30 minutes.

13. The method of claim 1, wherein the mesenchymal stem cells are obtained autogenously.

14. The method of claim 1, wherein administering the therapeutically effective amount of the second mixture to the patient is carried out by intravenous infusion.

15. A mesenchymal stem cell therapy composition useful for treating a patient suffering from one or more conditions, said composition produced by the steps of:
(a) obtaining mesenchymal stem cells;
(b) mixing the mesenchymal stem cells with a premixture comprising a predetermined quantity of an aqueous anticoagulant solution, a predetermined quantity of a dextrose solution, and a predetermined quantity of phosphate buffered saline, to form a first mixture; and,
(c) bathing said first mixture for a period of time in a glucose-containing sauce to form a second mixture;
wherein the one or more conditions the patient is suffering from are one or more of an autoimmune disease, acute traumatic soft tissue orthopedic injuries, or elevated liver enzymes from alcohol.

16. The mesenchymal stem cell therapy composition of claim 15, wherein the steps of producing the composition further comprise obtaining bone marrow concentrate that contains the mesenchymal stem cells.

17. The mesenchymal stem cell therapy composition of claim 16, wherein the steps of producing the composition further comprise aspirating bone marrow to obtain said bone marrow concentrate.

18. The mesenchymal stem cell therapy composition of claim 17, wherein aspirating the bone marrow further includes passing the bone marrow one or more times through one or more plugs of cancellous bone.

19. The mesenchymal stem cell therapy composition of claim 17, wherein the bone marrow concentrate is obtained from the bone marrow by centrifugation.

20. The mesenchymal stem cell therapy composition of claim 19, wherein obtaining the bone marrow concentrate is further filtered through a microfilter of a size that is effective in removing bone debris, aggregates, particulates, and/or non-cellular contaminants.

21. The mesenchymal stem cell therapy composition of claim 20, wherein the size of the microfilter is 18 μm.

22. The mesenchymal stem cell therapy composition of claim 15, wherein the bone marrow is aspirated from an ilium of a pelvis.

23. The mesenchymal stem cell therapy composition of claim 15, wherein the autoimmune disease is one or more than one of Parkinson's Disease, Multiple Sclerosis, Fibromyalgia, Rheumatoid Arthritis, and Crohn's Disease.

24. The mesenchymal stem cell therapy composition of claim 15, wherein the glucose-containing sauce comprises a glucose solution having a glucose concentration of from about 25% to about 75% by volume.

25. The mesenchymal stem cell therapy composition of claim 15, wherein the glucose solution includes glucose dissolved or suspended or emulsified in an aqueous medium, or in a mixture of an aqueous medium and one or more other art-recognized suitable media.

26. The mesenchymal stem cell therapy composition of claim 15, wherein the bathing period of time is between about 1 minute and about 5 minutes, between about 5 minutes and about 10 minutes, between about 10 minutes and about 15 minutes, between about 15 minutes and about 20 minutes, between about 20 minutes and about 25 minutes, or between about 25 minutes and about 30 minutes, or is longer than about 30 minutes.

27. The mesenchymal stem cell therapy composition of claim 15, wherein the mesenchymal stem cells are obtained autogenously.

28. The mesenchymal stem cell therapy composition of claim 15, wherein the composition is suitable for administering to the patient by intravenous infusion.
